(19) United States
(12) Patent Application Publication
(10) Pub. No.: US 2026/0125458 A1
(43) Pub. Date: May 7, 2026

(54) BISPECIFIC ANTI-VEGF/ANG2 ANTIBODY FORMULATION

(71) Applicant: Hoffmann-La Roche Inc., Little Falls, NJ (US)

(72) Inventors: Christian Freichel, Basel (CH); Claudia Mueller, Basel (CH); Robert Mueller, Basel (CH); Piotr Jan Szczesny, Basel (CH); Martin Worgull, Basel (CH); Christine Wurth, Basel (CH)

(73) Assignee: Hoffmann-La Roche Inc., Little Falls, NJ (US)

(21) Appl. No.: 19/397,576

(22) Filed: Nov. 21, 2025

Related U.S. Application Data
(63) Continuation of application No. 17/242,667, filed on Apr. 28, 2021, now Pat. No. 12,503,503.

(51) Int. Cl. C07K 16/22; A61M 5/178

(52) U.S. Cl. CPC C07K 16/22; A61M 5/178; C07K 2317/31; C07K 2317/526; C07K 2317/565; C07K 2317/94

(57) ABSTRACT

This invention relates to a pharmaceutical formulation of a bispecific anti-VEGF/ANG2 antibody, and a process for the preparation and uses of the formulation.

### CROSS REFERENCE TO RELATED APPLICATIONS

This application is a Continuation of U.S. application Ser. No. 17/242,667, filed Apr. 28, 2021, which is a continuation of International Application No. PCT/EP2019/079137, filed Oct. 25, 2019, which claims benefit of priority to EP Application Serial No. 18203104.7 filed Oct. 29, 2018, each of which is incorporated herein by reference in its entirety.

### SEQUENCE LISTING

This application contains a Sequence Listing which has been submitted electronically in XML format and is hereby incorporated by reference in its entirety. Said XML copy, created on Nov. 12, 2025, is named P35108-US-1_SL.xml and is 33,558 bytes in size.

### FIELD OF THE INVENTION

This invention relates to a liquid pharmaceutical formulation of bispecific antibodies against Angiopoietin-2 (ANG-2) and human vascular endothelial growth factor (VEGF, VEGF-A) (bispecific anti-VEGF/ANG2 antibodies) and a process for the preparation and uses of the formulation.

### BACKGROUND

Bispecific antibodies against Angiopoietin-2 (ANG-2) and human vascular endothelial growth factor (VEGF, VEGF-A) (bispecific anti-VEGF/ANG2 antibodies), are of therapeutic interest, in particular as medicaments for the treatment and prophylaxis of treatment of vascular diseases, including ocular vascular disease. Bispecific anti-VEGF/ANG2 antibodies are for example described in WO2010/040508, WO2011/117329 or WO2014/009465. These antibodies inhibit Vegf binding to the VEGF receptor and at the same time ANG-2 binding to Tie2.

Antibody molecules, as part of the group of protein pharmaceuticals, are very susceptible to physical and chemical degradation. Chemical degradation includes any process that involves modification of the protein via bond formation or cleavage, yielding a new chemical entity. A variety of chemical reactions is known to affect proteins. These reactions can involve hydrolysis including cleavage of peptide bonds as well as deamidation, isomerization, oxidation and decomposition. Physical degradation refers to changes in the higher order structure and includes denaturation, adsorption to surfaces, aggregation and precipitation. Protein stability is influenced by the characteristics of the protein itself, e.g. the amino acid sequence, the glycosylation pattern, and by external influences, such as temperature, solvent pH, excipients, interfaces, or shear rates. So, it is important to define the optimal formulation conditions to protect the protein against degradation reactions during manufacturing, storage and administration. (Manning, M. C., et al. (1989), “Stability of protein pharmaceuticals”, Pharm Res 6(11), 903-918; Zheng, J. Y., Janis, L. J. (2005), “Influence of pH, buffer species, and storage temperature on physicochemical stability of a humanized monoclonal antibody LA298”, Int. J. Pharmaceutics 308, 46-51). Stable liquid formulations of therapeutic antibodies are particularly difficult to obtain when the formulation should include antibodies in a high concentration.

It is therefore an object of the present invention to provide a liquid, in particular high concentration, formulation of a bispecific VEGF/ANG2 antibody with as few as necessary excipients, which enables the desired dosing and allows convenient intravitreal administration of the bispecific antibody through thin needles to a patient.

### SUMMARY

The present invention relates to a liquid pharmaceutical formulation of a bispecific anti-VEGF/ANG2 antibody, a method for the preparation and uses of the formulation. In particular, the pharmaceutical formulations of the present invention are for use in intravitreal administration for the treatment of ophthalmologic diseases like AMD and DME.

In one aspect, the invention refers to a liquid pharmaceutical formulation, comprising:
20 to 150 mg/ml of a bispecific anti-VEGF/ANG2 antibody comprising a constant heavy chain region of human IgG1 subclass
15 to 35 mM of sodium chloride
15 to 25 mM of a histidine acetate buffer
at a pH of 5.5±0.5; wherein
the bispecific anti-VEGF/ANG2 antibody is bivalent and comprises a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR 1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14,
and wherein
iii) the bispecific antibody comprises a constant heavy chain region of human IgG1 subclass comprising the mutations I253A, H310A, and H435A and the mutations L234A, L235A and P329G (numberings according to EU Index of Kabat.

In one embodiment the bispecific anti-VEGF/ANG2 antibody is bivalent and comprises the amino acid sequences of SEQ ID NO: 17, of SEQ ID NO: 18, of SEQ ID NO: 19, and of SEQ ID NO: 20.

In one embodiment the bispecific anti-VEGF/ANG2 antibody is faricimab.

In one embodiment the formulation further comprises
1 to 20 mM of at least one stabilizer.

In one embodiment the formulation further comprises
7.0 mM±2.0 mM methionine.

In one embodiment the formulation further comprises
0.01-0.07% (w/v) of a surfactant.

In one embodiment the formulation further comprises
0.04% (w/v)±0.02% (w/v) polysorbate 20.

In one embodiment the formulation further comprises
50-250 mM of a tonicity agent.

In one embodiment the formulation further comprises
160 mM±24 mM sucrose.

In one embodiment the formulation is essentially free of visible particles.

In one embodiment the formulation is a stable formulation.

In one embodiment the osmolality of the formulation is 300±100 mOsm/kg.

In one embodiment the formulation is for intravitreal administration.

In one aspect the formulation is for use in the treatment of an ocular vascular disease.

In one embodiment the ocular vascular disease is selected from the group consisting of diabetic retinopathy (DR), diabetic macular edema (DME), retinal vein occlusion (RVO), central retinal vein occlusion (CRVO), macular degeneration, wet age-related macular degeneration (wet AMD), retinopathy of prematurity (ROP), neovascular glaucoma, retinitis pigmentosa (RP), retinal angiomatous proliferation, macular telangiectasia, ischemic retinopathy, iris neovascularization, intraocular neovascularization, corneal neovascularization, retinal neovascularization, choroidal neovascularization, and retinal degeneration, in particular from the group consisting of diabetic retinopathy (DR), diabetic macular edema (DME), retinal vein occlusion (RVO), central retinal vein occlusion (CRVO), wet age-related macular degeneration (wet AMD).

One aspect of the invention is a method for the preparation of the pharmaceutical formulation according to the invention.

One aspect of the invention is a vial comprising the pharmaceutical formulation according to the invention.

One aspect of the invention is a prefilled syringe comprising the pharmaceutical formulation according to the invention.

One aspect of the invention is a lyophilized form of the liquid pharmaceutical formulation according to the invention.

The present invention provides a liquid pharmaceutical formulation of a bispecific anti-VEGF/ANG2 antibody (with IgG1 constant region) which has valuable properties useful for ophthalmologic use and intravitreal application: the formulation has low viscosity and low turbidity (even at high concentrations of e.g. about 120 mg/l), the formulation is stable and isotonic. This is achieved especially by the combination of 20 to 150 mg/ml (in particular of 100 to 140 mg/ml) of the bispecific anti-VEGF/ANG2 antibody comprising a constant heavy chain region of human IgG1 subclass as described herein, 15 to 35 mM of sodium chloride and 15 to 25 mM of a histidine buffer at a pH of 5.5±0.5.

### DESCRIPTION OF THE FIGURES

FIG. 1A Turbidity results of formulations from the pH/buffer screen part I. FIG. 1A compares the turbidity results of the formulations from the pH/buffer screen I (below the bars: first row: no. of formulation sample; second row: pH value; third row: buffer system; fourth row: ionic strength).

FIG. 1B Viscosity results of formulations from the pH/buffer screen part I. FIG. 1B compares the viscosity results of the formulations from the pH/buffer screen I (below the bars: first row: no. of formulation sample; second row: pH value; third row: buffer system; fourth row: ionic strength).

FIG. 2A Turbidity results of formulations from the pH/buffer screen part II (below the bars: first row: no. of formulation sample; second row pH value; third row; viscosity reducer absent -, NaCl or CaCl₂; fourth row: ionic strength).

FIG. 2B Viscosity results of formulations from the pH/buffer screen part II (below the bars: first row: no. of formulation sample; second row pH value; third row; viscosity reducer absent -, NaCl or CaCl₂; fourth row: ionic strength).

FIG. 3 Higher molecular weight species (HMW) of formulations from the pH/buffer screen part II at start and after 8 weeks storage at 5° C. and 25° C. (below the bars: first row: no. of formulation sample; second row pH value; third row: viscosity reducer absent -, NaCl or CaCl₂; fourth row: ionic strength).

FIG. 4 Levels of high molecular weight species (HMW) at initial and after physical stress (below the bars: first row: type of physical stress; second row: % surfactant; third row: no. of formulation sample).

FIG. 5A Turbidity of formulations from the excipient screen I (below the bars: first row: pH value with NaCl as viscosity reducer absence (−) or presence (+) of methionine as stabilizer; second row: no. of formulation sample).

FIG. 5B Viscosity of formulations from the excipient screen I (below the bars: first row: pH value with NaCl as viscosity reducer absence (−) or presence (+) of methionine as stabilizer; second row: no. of formulation sample).

FIG. 6 Levels of high molecular weight species (HMW) at initial and during storage at 5° C. (below the bars: first row: pH value with NaCl as viscosity reducer absence (−) or presence (+) of methionine as stabilizer; second row: no. of formulation sample).

FIG. 7 Levels of high molecular weight species (HMW) at initial and during storage at 25° C. (below the bars: first row: pH value with NaCl as viscosity reducer absence (−) or presence (+) of methionine as stabilizer; second row: no. of formulation sample).

FIG. 8A Levels of charged species (Main peak) at initial and during storage at 5° C. (below the bars: first row: pH value with NaCl as viscosity reducer absence (−) or presence (+) of methionine as stabilizer; second row: no. of formulation sample).

FIG. 8B Levels of charged species (Acidic peak) at initial and during storage at 5° C. (below the bars: first row: pH value with NaCl as viscosity reducer absence (−) or presence (+) of methionine as stabilizer; second row: no. of formulation sample).

FIG. 8C Levels of charged species (Basic peak) at initial and during storage at 5° C. (below the bars: first row: pH value with NaCl as viscosity reducer absence (−) or presence (+) of methionine as stabilizer; second row: no. of formulation sample).

FIG. 9A Levels of charged species (Main peak) at initial and during storage at 25° C. (below the bars: first row: pH value with NaCl as viscosity reducer; absence (−) or presence (+) of methionine as stabilizer; second row: no. of formulation sample.

FIG. 9B Levels of charged species (Acidic peak) at initial and during storage at 25° C. (below the bars: first row: pH value with NaCl as viscosity reducer; absence (−) or presence (+) of methionine as stabilizer; second row: no. of formulation sample.

FIG. 9C Levels of charged species (Basic peak) at initial and during storage at 25° C. (below the bars: first row: pH value with NaCl as viscosity reducer; absence (−) or presence (+) of methionine as stabilizer; second row: no. of formulation sample.

FIG. 10A Turbidity of optimized and reference formulation with a protein concentration of 120 mg/mL from the excipient screen II.

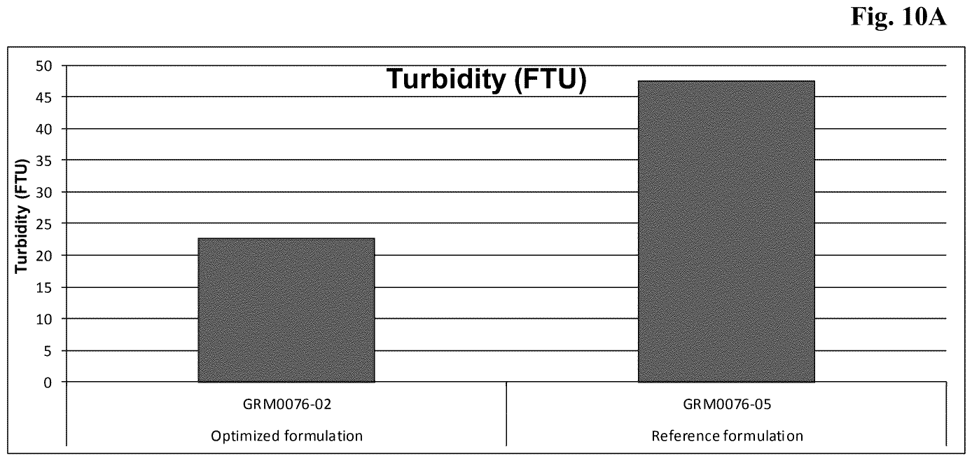

FIG. 10B Viscosity of optimized and reference formulation with a protein concentration of 120 mg/mL from the excipient screen II.

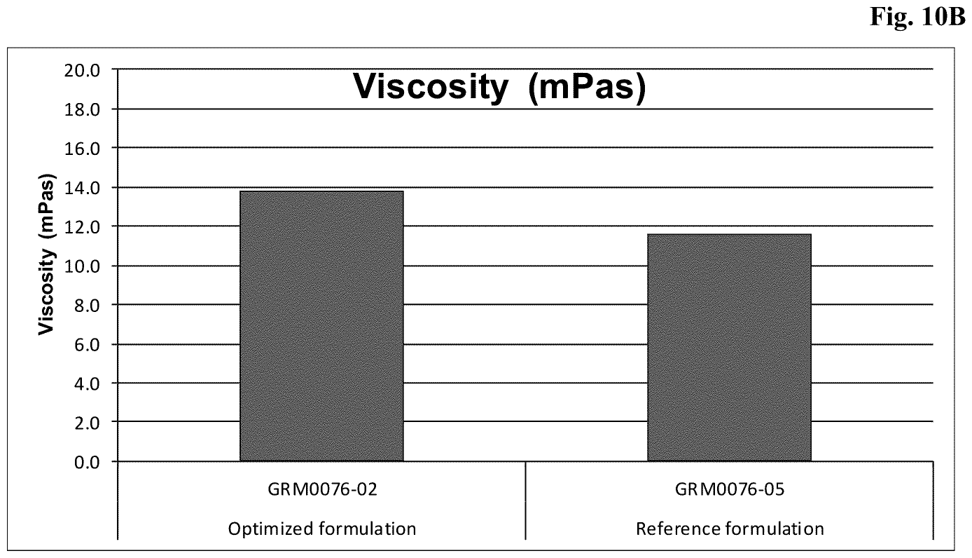

FIG. 11A Turbidity of optimized and reference formulation with a protein concentration of 30 mg/mL from the excipient screen II.

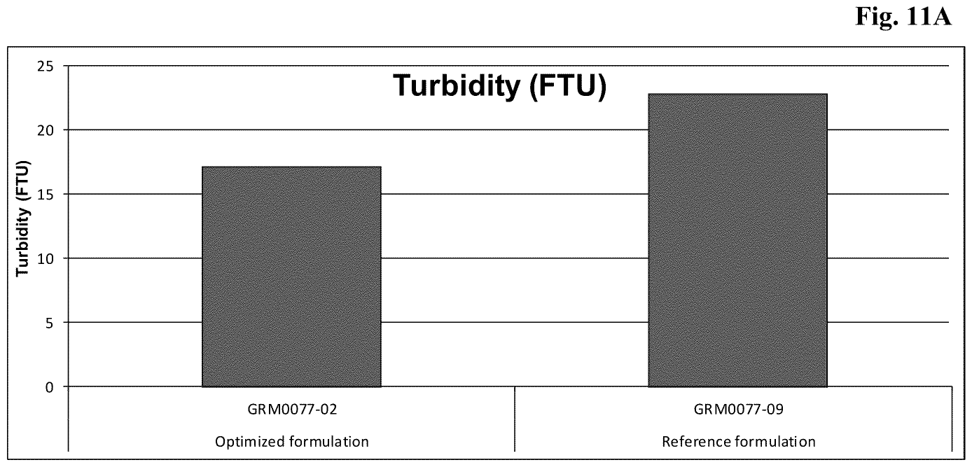

FIG. 11B Viscosity of optimized and reference formulation with a protein concentration of 30 mg/mL from the excipient screen II.

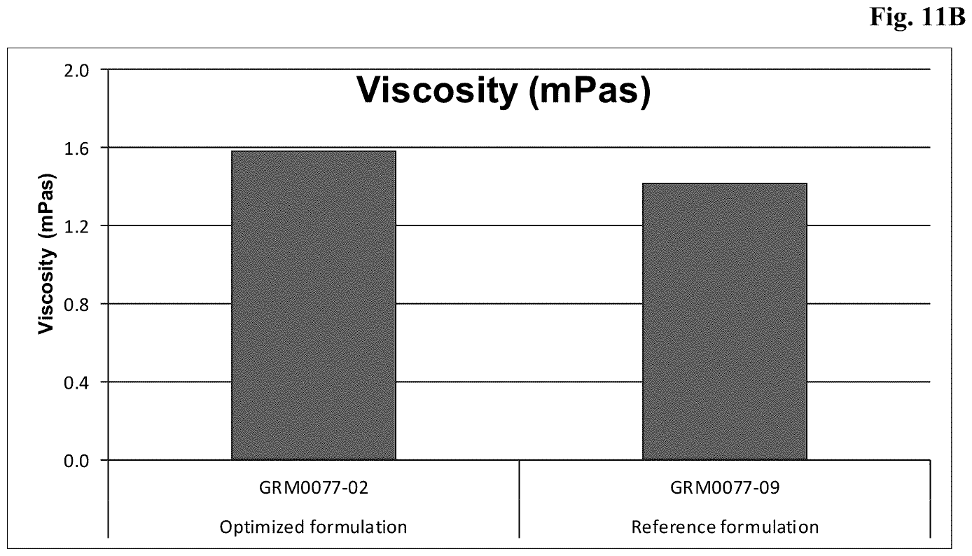

FIG. 12 Levels of high molecular weight species (HMW) at initial (left bar) and after 13 weeks storage at 5° C. (middle bar) and 25° C. (right bar) of optimized and reference formulation with a protein concentration of 120 mg/mL.

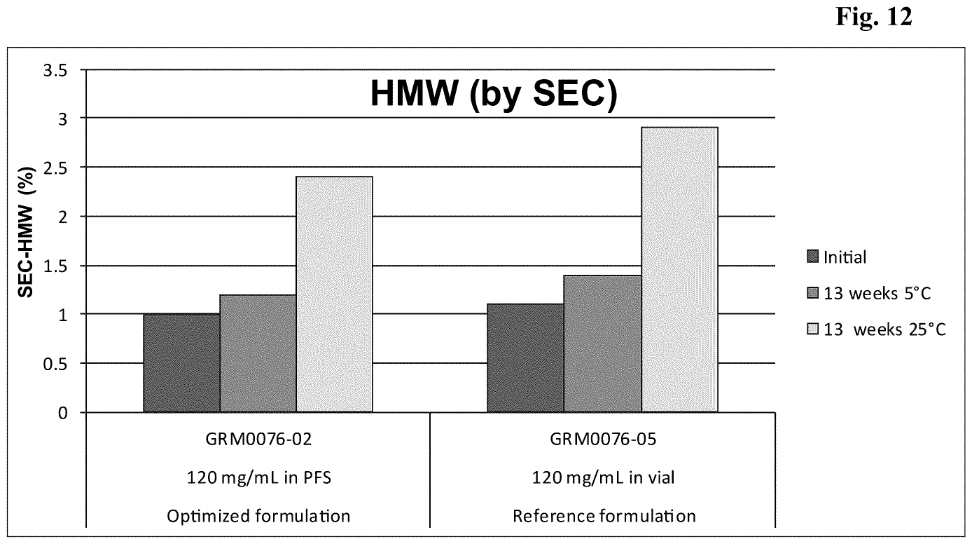

FIG. 13 Levels of high molecular weight species (HMW) at initial (left bar) and after 13 weeks storage at 5° C. (middle bar) and 25° C. (right bar) of optimized and reference formulation with a protein concentration of 30 mg/mL.

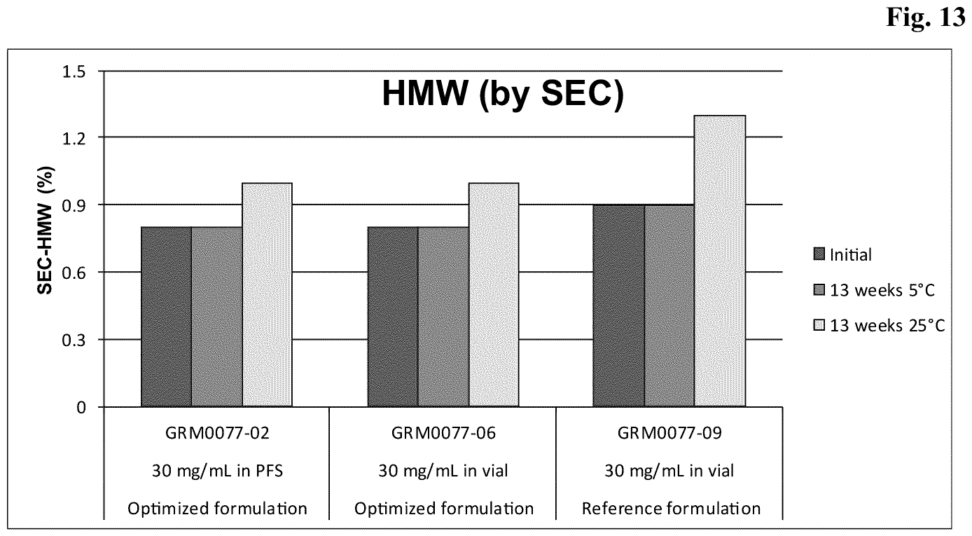

FIG. 14A Levels of charged species (Main peak) at initial (left bar) and after 13 weeks storage at 5° C. (middle bar) and 25° C. (right bar) of optimized and reference formulation.

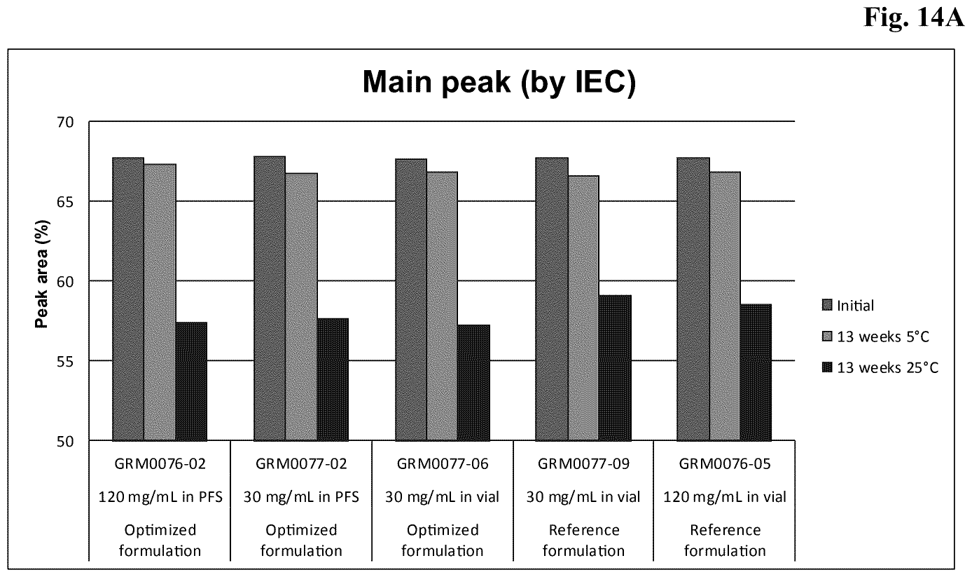

FIG. 14B Levels of charged species (Acidic peak) at initial (left bar) and after 13 weeks storage at 5° C. (middle bar) and 25° C. (right bar) of optimized and reference formulation.

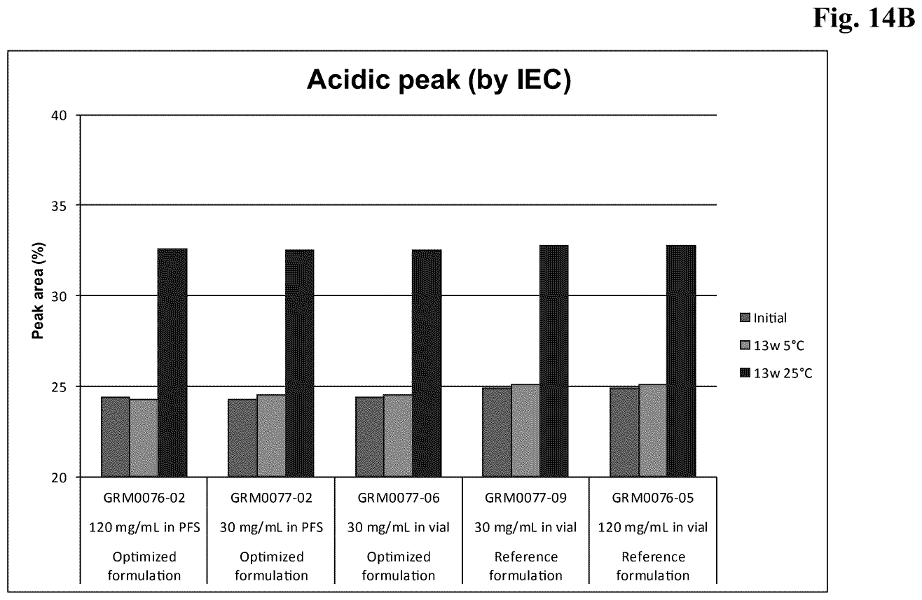

FIG. 14C Levels of charged species (Basic peak) at initial (left bar) and after 13 weeks storage at 5° C. (middle bar) and 25° C. (right bar) of optimized and reference formulation.

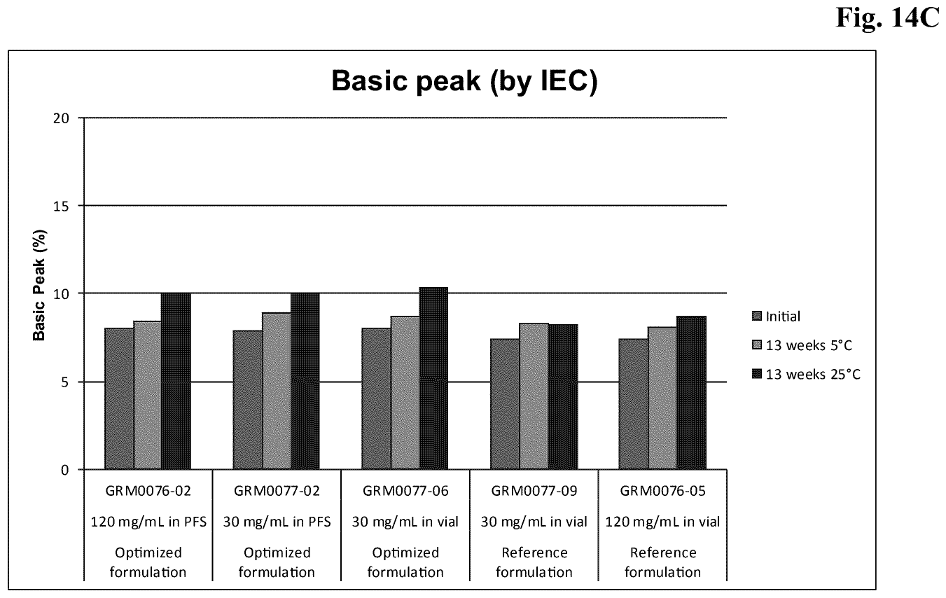

### DETAILED DESCRIPTION OF THE INVENTION

The present invention relates to a liquid pharmaceutical formulation comprising a bispecific anti-VEGF/ANG2 comprising a constant heavy chain region of human IgG1 subclass.

The term “pharmaceutical formulation” refers to preparations which are in such form as to permit the biological activity of the active ingredients to be unequivocally effective, and which contain no additional components which are toxic to the subjects to which the formulation is administered.

The term “liquid” as used herein in connection with the formulation according to the invention denotes a formulation which is liquid at least at a temperature between about 2° C. to about 35° C. (in one embodiment between about 2° C. to about 25° C.) under atmospheric pressure.

The concentration of the bispecific anti-VEGF/ANG2 antibody comprised in the pharmaceutical formulation is in the range of about 20 mg/ml to about 150 mg/ml, in particular the concentration 120 mg/ml±18 mg/ml, more particularly the concentration is 120 mg/ml±12 mg/ml. In another embodiment the concentration can be 30 mg/ml±4.5 mg/ml.

As used herein, “antibody” refers to a binding protein that comprises antigen-binding sites. The terms “binding site” or “antigen-binding site” as used herein denotes the region(s) of an antibody molecule to which a ligand actually binds. The term “antigen-binding site” comprises an antibody's heavy chain variable domains (VH) and an antibody's light chain variable domains (VL) (pair of VH/VL).

Antibody specificity refers to selective recognition of the antibody for a particular epitope of an antigen. Natural antibodies, for example, are monospecific.

“Bispecific antibodies” according to the invention are antibodies which have two different antigen-binding specificities. Antibodies of the present invention are specific for two different antigens, VEGF as first antigen and ANG-2 as second antigen.

The term “monospecific” antibody as used herein denotes an antibody that has one or more binding sites each of which binds to the same epitope of the same antigen.

The term “valent” as used within the current application denotes the presence of a specified number of binding sites in an antibody molecule. As such, the terms “bivalent”, “tetravalent”, and “hexavalent” denote the presence of two binding site, four binding sites, and six binding sites, respectively, in an antibody molecule. The bispecific antibodies according to the invention are preferably “bivalent”.

The terms “bispecific antibody which binds to human vascular endothelial growth factor (VEGF) and to human angiopoietin-2 (ANG-2)”, “bispecific anti-VEGF/ANG2 antibody” and bispecific <VEGF/ANG2> antibody” as used herein are interchangeable and refer to an antibody which has at least two different antigen-binding sites, a first one which binds to VEGF and a second one which binds to ANG2.

Bispecific anti-VEGF/ANG2 antibodies are e.g. described in WO2010/040508, WO2011/117329, WO2012/131078, WO2015/083978, WO2017/197199, and WO2014/009465. WO2014/009465 describes bispecific anti-VEGF/ANG2 antibodies especially designed for treatment of ocular vascular diseases. The bispecific anti-VEGF/ANG2 antibodies of WO2014/009465 (which is incorporated herein in its entirety) are especially useful in the treatment and treatment schedules of ocular vascular diseases as described herein. In particular, anti-VEGF/ANG2 antibody CrossMAb VEGFang2-0016 as described in WO2014/009465 which is also described as faricimab (in World Health Organization (2017). “International Nonproprietary Names for Pharmaceutical Substances (INN). Proposed INN: List 118” WHO Drug Information. 31 (4)) is a preferred bispecific anti-VEGF/ANG2 antibody of the present invention.

In one embodiment the bispecific antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14,
and wherein
iii) the bispecific antibody comprises a constant heavy chain region of human IgG1 subclass comprising the mutations 1253A, H310A, and H435A and the mutations L234A, L235A and P329G (numberings according to EU Index of Kabat).

In one embodiment such bispecific anti-VEGF/ANG2 antibody is bivalent.

In one embodiment such bispecific, bivalent anti-VEGF/ANG2 antibody is characterized in that
i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.

In one aspect of the invention such bispecific, bivalent antibody according to the invention is characterized in comprising
a) the heavy chain and the light chain of a first full length antibody that specifically binds to VEGF;
b) the modified heavy chain and modified light chain of a second full length antibody that specifically binds to ANG-2, wherein the constant domains CL and CH1 are replaced by each other.

This bispecific, bivalent antibody format for the bispecific antibody specifically binding to human vascular endothelial growth factor (VEGF) and human angiopoietin-2 (ANG-2) is described in WO 2009/080253 (including Knobs-into-Holes modified CH3 domains). The antibodies based on this bispecific, bivalent antibody format are named CrossMAbs.

In one embodiment such bispecific, bivalent anti-VEGF/ANG2 antibody is characterized in comprising
a) as heavy chain of the first full length antibody the amino acid sequence of SEQ ID NO: 17, and as light chain of the first full length antibody the amino acid sequence of SEQ ID NO: 18, and
b) as modified heavy chain of the second full length antibody the amino acid sequence of SEQ ID NO: 19, and as modified light chain of the second full length antibody the amino acid sequence of SEQ ID NO: 20.
In one embodiment such bispecific, bivalent anti-VEGF/ANG2 antibody is characterized in comprising the amino acid sequences of SEQ ID NO: 17, of SEQ ID NO: 18, of SEQ ID NO: 19, and of SEQ ID NO: 20

Accordingly, one embodiment of the invention is a bispecific, bivalent antibody comprising a first antigen-binding site that specifically binds to human VEGF and a second antigen-binding site that specifically binds to human ANG-2, characterized in comprising the amino acid sequences of SEQ ID NO: 17, of SEQ ID NO: 18, of SEQ ID NO: 19, and of SEQ ID NO: 20.

In one embodiment the CH3 domains of the bispecific, bivalent antibody according to the invention is altered by the “knob-into-holes” technology which is described in detail with several examples in e.g. WO 96/027011, Ridgway J. B., et al., Protein Eng 9 (1996) 617-621; and Merchant, A. M., et al., Nat Biotechnol 16 (1998) 677-681. In this method the interaction surfaces of the two CH3 domains are altered to increase the heterodimerization of both heavy chains containing these two CH3 domains. Each of the two CH3 domains (of the two heavy chains) can be the “knob”, while the other is the “hole”. The introduction of a disulfide bridge stabilizes the heterodimers (Merchant, A. M, et al., Nature Biotech 16 (1998) 677-681; Atwell, S., et al. J. Mol. Biol. 270 (1997) 26-35) and increases the yield.

In a preferred aspect of the invention the bispecific anti-VEGF/ANG2 antibodies according to the invention are characterized in that
the CH3 domain of one heavy chain and the CH3 domain of the other heavy chain each meet at an interface which comprises an original interface between the antibody CH3 domains;
wherein said interface is altered to promote the formation of the bispecific antibody, wherein the alteration is characterized in that:
a) the CH3 domain of one heavy chain is altered,
so that within the original interface the CH3 domain of one heavy chain that meets the original interface of the CH3 domain of the other heavy chain within the bispecific antibody,
an amino acid residue is replaced with an amino acid residue having a larger side chain volume, thereby generating a protuberance within the interface of the CH3 domain of one heavy chain which is positionable in a cavity within the interface of the CH3 domain of the other heavy chain
and
b) the CH3 domain of the other heavy chain is altered,
so that within the original interface of the second CH3 domain that meets the original interface of the first CH3 domain within the bispecific antibody
an amino acid residue is replaced with an amino acid residue having a smaller side chain volume, thereby generating a cavity within the interface of the second CH3 domain within which a protuberance within the interface of the first CH3 domain is positionable.

Thus the bispecific anti-VEGF/ANG2 antibodies for use described herein are preferably characterized in that
the CH3 domain of the heavy chain of the full length antibody of a) and the CH3 domain of the heavy chain of the full length antibody of b) each meet at an interface which comprises an alteration in the original interface between the antibody CH3 domains;
wherein i) in the CH3 domain of one heavy chain
an amino acid residue is replaced with an amino acid residue having a larger side chain volume, thereby generating a protuberance within the interface of the CH3 domain of one heavy chain which is positionable in a cavity within the interface of the CH3 domain of the other heavy chain
and wherein
ii) in the CH3 domain of the other heavy chain
an amino acid residue is replaced with an amino acid residue having a smaller side chain volume, thereby generating a cavity within the interface of the second CH3 domain within which a protuberance within the interface of the first CH3 domain is positionable.

Preferably said amino acid residue having a larger side chain volume is selected from the group consisting of arginine (R), phenylalanine (F), tyrosine (Y), tryptophan (W).

Preferably said amino acid residue having a smaller side chain volume is selected from the group consisting of alanine (A), serine(S), threonine (T), valine (V).

In one aspect of the invention both CH3 domains are further altered by the introduction of cysteine (C) as amino acid in the corresponding positions of each CH3 domain such that a disulfide bridge between both CH3 domains can be formed.

In one embodiment, the bispecific antibody comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M, et al., Nature Biotech 16 (1998) 677-681) e.g. by introducing a S354C mutation into one CH3 domain and a Y349C mutation into the other CH3 domain.

In a another preferred embodiment the bispecific antibody comprises S354C and T366W mutations in one of the two CH3 domains and Y349C, T366S, L368A, Y407V mutations in the other of the two CH3 domains In a another preferred embodiment the bispecific antibody comprises Y349C, T366W mutations in one of the two CH3 domains and S354C, T366S, L368A, Y407V mutations in the other of the two CH3 domains (the additional Y349C or S354C mutation in one CH3 domain and the additional S354C or Y349C mutation in the other CH3 domain forming a interchain disulfide bridge) (numbering always according to EU index of Kabat (Kabat, E. A., et al., Sequences of Proteins of Immunological Interest, 5th ed., Public Health Service, National Institutes of Health, Bethesda, MD (1991)).

Other techniques for CH3-modifications to enforce the heterodimerization are contemplated as alternatives of the invention and described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291.

In one embodiment the heterodimerization approach described in EP 1 870 459A1 is used alternatively. This approach is based on the introduction of substitutions/mutations of charged amino acids with the opposite charge at specific amino acid positions of the in the CH3/CH3 domain interface between both heavy chains. One preferred embodiment for said multispecific antibodies are amino acid R409D and K370E mutations in the CH3 domain of one heavy chain and amino acid D399K and E357K mutations in the CH3 domain of the other heavy chain of the multispecific antibody (numberings according to Kabat EU index).

In another embodiment said multispecific antibody comprises an amino acid T366W mutation in the CH3 domain of the “knobs chain” and amino acid T366S, L368A and Y407V mutations in the CH3 domain of the “hole chain”; and additionally comprises amino acid R409D and K370E mutations in the CH3 domain of the “knobs chain” and amino acid D399K and E357K mutations in the CH3 domain of the “hole chain”.

In one embodiment the heterodimerization approach described in WO2013/157953 is used alternatively. In one embodiment the CH3 domain of one heavy chain comprises an amino acid T366K mutation and the CH3 domain of the other heavy chain comprises an amino acid L351D mutation. In a further embodiment the CH3 domain of the one heavy chain further comprises an amino acid L351K mutation. In a further embodiment the CH3 domain of the other heavy chain further comprises an amino acid mutation selected from Y349E, Y349D and L368E (in one embodiment L368E).

In one embodiment the heterodimerization approach described in WO2012/058768 is used alternatively. In one embodiment the CH3 domain of one heavy chain comprises amino acid L351Y and Y407A mutations and the CH3 domain of the other heavy chain comprises amino acid T366A and K409F mutations. In a further embodiment the CH3 domain of the other heavy chain further comprises an amino acid mutation at position T411, D399, S400, F405, N390 or K392. In one embodiment said amino acid mutation is selected from the group consisting of
a) T411N, T411R, T411Q, T411K, T411D, T411E and T411W,
b) D399R, D399W, D399Y and D399K,
c) S400E, S400D, S400R and S400K,
d) F405I, F405M, F405T, F405S, F405V and F405W,
e) N390R, N390K and N390D,
f) K392V, K392M, K392R, K392L, K392F and K392E.

In a further embodiment the CH3 domain of one heavy chain comprises amino acid L351Y and Y407A mutations and the CH3 domain of the other heavy chain comprises amino acid T366V and K409F mutations. In a further embodiment the CH3 domain of one heavy chain comprises an amino acid Y407A mutation and the CH3 domain of the other heavy chain comprises amino acid T366A and K409F mutations. In a further embodiment the CH3 domain of the other heavy chain further comprises amino acid K392E, T411E, D399R and S400R mutations.

In one embodiment the heterodimerization approach described in WO2011/143545 is used alternatively. In one embodiment the amino acid modification according to WO2011/143545 is introduced in the CH3 domain of the heavy chain at a position selected from the group consisting of 368 and 409.

In one embodiment the heterodimerization approach described in WO2011/090762 which also uses the knob-into-hole technology described above is used alternatively. In one embodiment the CH3 domain of one heavy chain comprises an amino acid T366W mutation and the CH3 domain of the other heavy chain comprises an amino acid Y407A mutation. In one embodiment the CH3 domain of one heavy chain comprises an amino acid T366Y mutation and the CH3 domain of the other heavy chain comprises an amino acid Y407T mutation.

In one embodiment the multispecific antibody is of IgG2 isotype and the heterodimerization approach described in WO2010/129304 is used alternatively.

In one embodiment the heterodimerization approach described in WO2009/089004 is used alternatively. In one embodiment the CH3 domain of one heavy chain comprises an amino acid substitution of K392 or N392 with a negatively-charged amino acid (in one embodiment glutamic acid (E) or aspartic acid (D); in a further embodiment a K392D or N392D mutation) and the CH3 domain of the other heavy chain comprises an amino acid substitution of D399, E356, D356, or E357 with a positively-charged amino acid (in one embodiment Lysine (K) or arginine (R), in a further embodiment a D399K, E356K, D356K or E357K substitution; and in an even further embodiment a D399K or E356K mutation). In a further embodiment the CH3 domain of the one heavy chain further comprises an amino acid substitution of K409 or R409 with a negatively-charged amino acid (in one embodiment glutamic acid (E) or aspartic acid (D); in a further embodiment a K409D or R409D mutation). In a further embodiment the CH3 domain of the one heavy chain further or alternatively comprises an amino acid substitution of K439 and/or K370 with a negatively-charged amino acid (in one embodiment glutamic acid (E) or aspartic acid (D)).

In one embodiment the heterodimerization approach described in WO2007/147901 is used alternatively. In one embodiment the CH3 domain of one heavy chain comprises amino acid K253E, D282K and K322D mutations and the CH3 domain of the other heavy chain comprises amino acid D239K, E240K and K292D mutations.

In one embodiment the heterodimerization approach described in WO2007/110205 is used alternatively.

In one preferred embodiment such bispecific anti-VEGF/ANG2 antibody is bivalent.

In one embodiment the bispecific, bivalent antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR 1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14,
and wherein
iii) the bispecific antibody comprises a constant heavy chain region of human IgG1 subclass comprising the mutations I253A, H310A, and H435A and the mutations L234A, L235A and P329G (numberings according to EU Index of Kabat; and wherein
iv) in the constant heavy chain region a T366W mutation is comprised in one CH3 domain and T366S, L368A, Y407V mutations are comprised the other CH3 domain (numberings according to EU Index of Kabat).

In one embodiment the bispecific, bivalent antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14,
and wherein
iii) the bispecific antibody comprises a constant heavy chain region of human IgG1 subclass comprising the mutations 1253A, H310A, and H435A and the mutations L234A, L235A and P329G (numberings according to EU Index of Kabat; and wherein
iv) in the constant heavy chain region a S354C and T366W mutations are comprised in one CH3 domain and Y349C, T366S, L368A and Y407V mutations are comprised the other CH3 domain (numberings according to EU Index of Kabat).

In one embodiment such bispecific, bivalent anti-VEGF/ANG2 is characterized in comprising the amino acid sequences of SEQ ID NO: 17, of SEQ ID NO: 18, of SEQ ID NO: 19, and of SEQ ID NO: 20.

Accordingly, one embodiment of the invention the antibody is a bispecific, bivalent antibody comprising a first antigen-binding site that specifically binds to human VEGF and a second antigen-binding site that specifically binds to human ANG-2, characterized in comprising the amino acid sequences of SEQ ID NO: 17, of SEQ ID NO: 18, of SEQ ID NO: 19, and of SEQ ID NO: 20.

In one preferred embodiment such bispecific anti-VEGF/ANG2 antibody is faricimab.

The term “VEGF” as used herein refers to human vascular endothelial growth factor (VEGF/VEGF-A,) the 165-amino acid human vascular endothelial cell growth factor (amino acid 27-191 of precursor sequence of human VEGF165: SEQ ID NO: 25; amino acids 1-26 represent the signal peptide), and related 121, 189, and 206 vascular endothelial cell growth factor isoforms, as described by Leung, D. W., et al., Science 246 (1989) 1306-9; Houck et al., Mol. Endocrin. 5 (1991) 1806-1814; Keck, P. J., et al., Science 246 (1989) 1309-12 and Connolly, D. T., et al., J. Biol. Chem. 264 (1989) 20017-24; together with the naturally occurring allelic and processed forms of those growth factors. VEGF is involved in the regulation of normal and abnormal angiogenesis and neovascularization associated with tumors and intraocular disorders (Ferrara, N., et al., Endocr. Rev. 18 (1997) 4-25; Berkman, R. A., et al., J. Clin. Invest. 91 (1993) 153-159; Brown, L. F., et al., Human Pathol. 26 (1995) 86-91; Brown, L. F., et al., Cancer Res. 53 (1993) 4727-4735; Mattern, J., et al., Brit. J. Cancer. 73 (1996) 931-934; and Dvorak, H. F., et al., Am. J. Pathol. 146 (1995) 1029-1039). VEGF is a homodimeric glycoprotein that has been isolated from several sources and includes several isoforms. VEGF shows highly specific mitogenic activity for endothelial cells. A VEGF antagonist/inhibitor inhibits binding of VEGF to its receptor VEGFR. Known VEGF antagonist/inhibitors include bispecific anti-VEGF/ANG2 antibodies as described in WO2014/009465.

The term “ANG-2” as used herein refers to human angiopoietin-2 (ANG-2) (alternatively abbreviated with ANGPT2 or ANG2) (SEQ ID NO: 24) which is described e.g. in Maisonpierre, P. C., et al, Science 277 (1997) 55-60 and Cheung, A. H., et al., Genomics 48 (1998) 389-91. The angiopoietins-1 and -2 were discovered as ligands for the Ties, a family of tyrosine kinases that is selectively expressed within the vascular endothelium (Yancopoulos, G. D., et al., Nature 407 (2000) 242-48). There are now four definitive members of the angiopoietin family. Angiopoietin-3 and -4 (Ang-3 and Ang-4) may represent widely diverged counterparts of the same gene locus in mouse and man (Kim, I., et al., FEBS Let, 443 (1999) 353-56; Kim, I., et al., J Biol Chem 274 (1999) 26523-28). ANG-1 and ANG-2 were originally identified in tissue culture experiments as agonist and antagonist, respectively (see for ANG-1: Davis, S., et al., Cell 87 (1996) 1161-69; and for ANG-2: Maisonpierre, P. C., et al., Science 277 (1997) 55-60). All of the known angiopoietins bind primarily to its receptor TIE2, and both Ang-1 and -2 bind to TIE2 with an affinity of 3 nM (Kd) (Maisonpierre, P. C., et al., Science 277 (1997) 55-60). An ANG2 antagonist/inhibitor inhibits binding of ANG2 to its receptor TIE2. Known ANG2 antagonist/inhibitors include bispecific anti-VEGF/ANG2 antibodies as described in WO2014/009465.

The antigen-binding sites of the bispecific antibody of the invention contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences.

The antibodies of the invention comprise immunoglobulin constant regions derived from human origin of immunoglobulin class IgG1.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are of particular interest. Other forms of “chimeric antibodies” encompassed by the present invention are those in which the constant region has been modified or changed from that of the original antibody to generate the desired properties according to the invention, especially in regard to C1q binding and/or Fc receptor (FcR) binding. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See e.g. Morrison, S. L., et al., Proc. Natl. Acad. Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See e.g. Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric antibodies. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to C1q binding and/or FcR binding, e.g. by “class switching” i.e. change or mutation of Fc parts (e.g. from IgG1 to IgG4 and/or IgG1/IgG4 mutation.).

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The “variable region” (variable region of a light chain (V_{L}), variable region of a heavy chain (V_{H})) or “variable domain” as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention. The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, MD (1991) and/or those residues from a “hypervariable loop”.

The term “epitope” includes any polypeptide determinant capable of specific binding to an antibody. In certain embodiments, epitope determinant includes chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

The term “full length antibody” denotes an antibody consisting of two “full length antibody heavy chains” and two “full length antibody light chains”. A “full length antibody heavy chain” is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CH1, HR, CH2 and CH3. A “full length antibody light chain” is a polypeptide consisting in N-terminal to C-terminal direction of an antibody light chain variable domain (VL), and an antibody light chain constant domain (CL), abbreviated as VL-CL. The antibody light chain constant domain (CL) can be kappa or lambda. The two full length antibody chains are linked together via inter-polypeptide disulfide bonds between the CL domain and the CHI domain and between the hinge regions of the full length antibody heavy chains. Examples of typical full length antibodies are natural antibodies like IgG (e.g. IgG1 and IgG2), IgM, IgA, IgD, and IgE. The full length antibodies according to the invention can be from a single species e.g. human, or they can be chimerized or humanized antibodies. The full length antibodies according to the invention comprise two antigen binding sites each formed by a pair of VH and VL, which both specifically bind to the same antigen. The C-terminus of the heavy or light chain of said full length antibody denotes the last amino acid at the C-terminus of said heavy or light chain. The N-terminus of the heavy or light chain of said full length antibody denotes the last amino acid at the N-terminus of said heavy or light chain.

The term “constant region” or “constant domains” as used within the current applications denotes the sum of the domains of an antibody other than the variable region. The constant region is not involved directly in binding of an antigen, but exhibits various effector functions. Depending on the amino acid sequence of the constant region of their heavy chains, antibodies are divided in the classes: IgA, IgD, IgE, IgG and IgM, and several of these may be further divided into subclasses, such as IgG1, IgG2, IgG3, and IgG4, IgA1 and IgA2. The heavy chain constant regions that correspond to the different classes of antibodies are called alpha, delta, epsilon, gamma, and micro, respectively. The light chain constant regions which can be found in all five antibody classes are called kappa and lambda.

The term “constant region derived from human origin” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., (see e.g. Johnson, G., and Wu, T. T., Nucleic Acids Res. 28 (2000) 214-218; Kabat, E. A., et al, Proc. Natl. Acad. Sci. USA 72 (1975) 2785-2788).

The term constant heavy chain domain (or region) as used herein defines a C-terminal region of an immunoglobulin heavy chain that contains at least a portion of the constant heavy chain region.

The term includes native sequences of the constant heavy chain domains and variant constant heavy chain domains. Variant constant heavy chain domains include e.g. mutations in the constant domain which are used to foster the heterodimerization as describe above for the knobs into hole technology. Also other mutations like e.g. L234A (Leu235Ala), L235A (Leu234Ala) and P329G (Pro329Gly) can be included as constant domains with such mutations have a reduced FcR binding (especially they show no more binding to FcRgammaI, FcRgammaII and FcRgammaIII). This especially useful to reduce potential side effects like e.g. thrombosis (Meyer, T., et al., J. Thromb. Haemost. 7 (2009) 171-81). In addition e.g. also the mutations I253A, H310A, and H435A (numbering according to EU Index of Kabat) can be included in the constant domain as constant domains with such mutations have a reduced FcRn one or two mutations) or eliminated FcRn binding (all 3 mutations).

In one aspect, a human IgG heavy chain constant region extends from alanine118 (A118) (numbering according to EU index of Kabat) to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the constant heavy chain domain may or may not be present. Amino acid sequences of heavy chains including the constant heavy chain domain are denoted herein with C-terminal glycine-lysine dipeptide if not indicated otherwise.

A “stable” formulation is one in which the protein therein, e.g. the antibody, essentially retains its physical and chemical stability and thus its biological activity upon storage; e.g. wherein the high molecular weight content (HMW) of the bispecific antibody in the pharmaceutical formulation is below 10% after 8 weeks at 25° C. (in one embodiment below 5%, in one embodiment below 2.5%). In one embodiment the high molecular weight content (HMW) of the bispecific antibody in the pharmaceutical formulation is below 10% after 52 weeks at 25° C. (in one embodiment below 5%).

In one embodiment a “stable liquid pharmaceutical formulation” is a liquid formulation with no significant changes observed at a refrigerated temperature (2-8° C.) for at least 12 months, particularly 2 years, and more particularly 3 years. The criteria for stability are the following: no more than 10%, particularly 5%, of antibody monomer is degraded as measured by size exclusion chromatography (SEC-HPLC). Furthermore, the solution is colorless or clear to slightly opalescent by visual analysis. The protein concentration of the formulation has no more than +/−10% change. No more than 10%, particularly 5% of aggregation is formed. The stability is measured by methods known in the art such UV spectroscopy, size exclusion chromatography (SEC-HPLC), Ion-Exchange Chromatography (IE-HPLC), turbidimetry and visual inspection.

### Turbidity (in FTU (=Formazine Turbidity Unit))

The turbidity of a pharmaceutical formulation can be determined on a turbidimeter (e.g. on a Hach 2100 AN turbidimeter according to Ph. Eur. 2.2.1 (Clarity and degree of opalescence of Liquids). A sample volume of approximately 2 mL sample solution is transferred into a 11 mm inner diameter glass cuvette and m. The glass cuvette is placed into the turbidimeter and the turbidity is measured against a calibration curve of the reference suspensions 1 FTU, 3 FTU, 10 FTU, 20 FTU and 100 FTU.

Viscosity (in mPa)

The viscosity of the formulation samples of a pharmaceutical formulation can be determined on a rheometer (e.g. an Anton Paar Physica MCR 301 rotational rheometer with a 25 mm-0.5° cone at a shear rate of 1000 s⁻¹ and a temperature of 20° C.).

### Visible Particles

The vial samples are visually inspected on respective inspection machine (e.g. a Seidenader inspection machine V90-T with help of a 2× magnifier lens. The illuminating light sources L1, L2 and L3 were adjusted to setting 5. The vial samples were inspected during a rotational movement for the presence of particles. The formation of visible particles is not acceptable for an intravitreal injection according to the requirements of USP-NF <790>, which is essentially free of visible particles. USP-NF <790> provides that the ‘⋅essentially free” standard is achieved when parenteral drugs are inspected and no more than a specified number of units are observed to contain visible particulates. More specifically, for parenteral drugs subject to 100% inspection, the “essentially free” standard is met when a batch meets an acceptable quality level (AQL) of 0.65% or lower. And if it becomes necessary to evaluate product that has been shipped to customers (e.g., because of a complaint or regulatory concern), a firm can sample and inspect 20 units. If no particles are observed in the sample, the batch is considered “essentially free” of visible particulates.

Protein Concentration (in mg/ml).

The protein concentration of the formulation samples was measured by ultraviolet (UV) light absorption on an UV/Vis Photometer Lambda™ 35 from Perkin Elmer. The formulation samples were diluted with a 20 mM L-histidine-acetate buffer solution pH 5.5 to a protein concentration of approximately 0.5 mg/mL and filled into a measurement cuvette with a thickness of 1 cm. The UV absorption of the measurement cuvette was measured at wavelengths at 280 and 320 nm.

The protein concentration was calculated from the measured UV light absorptions at 280 (A280) and 320 nm (A320), the extinction coefficient (E) of 1.70 mL/(mg×cm), the thickness (d) of 1 cm and dilution factor (DF) corresponding to the actual dilution according to the following equation:

$$Protein concentration in mg / mL = \frac{( A 280 - A 320 )}{( E \times d )} \times DF$$

pH

The pH of the formulations samples was determined by potentiometry with a glass electrode.

### Ionic Strength

The dimensionless ionic strength I of formulations is calculated according to Equation 1:

$$I = {\sum }_{i} {z}_{i}^{2} ( {b}_{i} / {b}^{θ} ) \qquad Equation 1$$

In this expression z is the charge number of an ion I (positive for cations and negative for anions) bi is its molality. b^{θ} corresponds to 1 mol/kg and is required to make I dimensionless (Physical Chemistry, P. Atkins, J. de Paula, Oxford Press Nineth edition, p. 194).

The molality of charged buffer species was calculated using Henderson-Hasselbalch equation (Methods in Enzymology—Guide to Protein Purification, Volume 182, M. P. Deutscher, Academic Press, Inc., 1990, p. 24ff).

### Osmolality

The osmolality of the formulation samples was measured on an Osmomat® 030 3P osmometer from Gonotec® according to the principle of freezing point depression.

### Surfactants

The pharmaceutical formulation of the present invention comprises a surfactant to reduce aggregation of the antibodies and particle formation. The term “surfactant” as used herein denotes a pharmaceutically acceptable excipient which is used to protect protein formulations against mechanical stresses like agitation and shearing. Examples of pharmaceutically acceptable surfactants include polyoxyethylenesorbitan fatty acid esters (Tween®), polyoxyethylene alkyl ethers (for example those sold under the trademark Brij™) and polyoxyethylene-polyoxypropylene copolymer (Poloxamer, Pluronic®).

Preferably, the surfactant is a polyoxyethylenesorbitan-fatty acid ester or a polaxamer. Examples of polyoxyethylenesorbitan-fatty acid esters are polysorbate 20 (sold under the trademark Tween® 20) and polysorbate 80 (sold under the trademark Tween® 80). The preferred polyoxyethylenesorbitan-fatty acid is polysorbate 20.

The above mentioned surfactants are generally used in a concentration of 0.01% (w/v) or higher, e.g. 0.01 to about 0.09% (w/v). The surfactant in a pharmaceutical composition of the present invention are in particular used in the range of about 0.02% to about 0.06% (w/v), more particular in the range of about 0.03% to about 0.05% (w/v), even more particularly in a concentration of about 0.04% (w/v).

The term “poloxamer” as used herein includes a polyoxyethylene-polyoxypropylene triblock copolymer composed of a central hydrophobic chain of polyoxypropylene flanked by two hydrophilic chains of polyoxyethylene known as poloxamer 188, sold under the trade name PLURONIC® F68 by BASF (Parsippany, N.J.). Other poloxamers which may be utilized in the formulations of the present invention include poloxamer 403 (sold as PLURONIC® P123), poloxamer 407 (sold as PLURONIC® P127), poloxamer 402 (sold as PLURONIC® P122), poloxamer 181 (sold as PLURONIC® L61), poloxamer 401 (sold as PLURONIC® L121), poloxamer 185 (sold as PLURONIC® P65), and poloxamer 338 (sold as PLURONIC® F108).

### Buffers

The term “buffer” as used herein denotes a pharmaceutically acceptable excipient, which stabilizes the pH of a pharmaceutical preparation. Suitable buffers are well known in the art and can be found in the literature. Typical pharmaceutically acceptable buffers for intravitreal administrations comprise but are not limited to histidine-buffers, citrate-buffers, succinate-buffers, acetate-buffers, phosphate-buffers or mixtures thereof. In this context buffers of particular interest comprise L-histidine (“histidine buffer”) or mixtures of L-histidine and L-histidine hydrochloride with pH adjustment with an acid or a base known in the art. Buffers of particular interest comprise L-histidine (“histidine buffer”) in particular L-histidine with pH adjustment with acetic acid (e.g. 30%) or hydrochloride. The abovementioned buffers are generally used in a concentration of about 2 mM to about 200 mM or about 5 mM to about 100 mM, particularly in a concentration of about 10 mM to about 30 mM or about 15 mM to about 20 mM and more particularly of about 20 mM. Independently from the buffer used, the pH can be adjusted to a value in the range from 4.5 to 7.0 and particularly to a value in the range from 5.0 to 6.0 and most particularly to pH 5.5±0.2 with an acid or a base known in the art, e.g. acetic acid, hydrochloric acid, phosphoric acid, sulfuric acid and citric acid, sodium hydroxide and potassium hydroxide, in particular with acetic acid. A buffer of particular interest is a histidine (L-histidine) acetate buffer in concentration of 15-25 mM (in one embodiment 20 mM±3 mM, in particular 20 mM±2 mM) at a pH of 5.5±0.5 (in one embodiment at a pH of 5.5±0.3; in particular, at a pH of 5.5±0.2)

### Stabilizers

The term “stabilizer” denotes a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland et al. (1993), Crit Rev Ther Drug Carrier Syst 10(4):307-77, Wang (1999) Int J Pharm 185(2):129-88, Wang (2000) Int J Pharm 203(1-2):1-60 and Chi et al. (2003) Pharm Res 20(9):1325-36. Stabilizers include but are not limited to sugars, amino acids, polyols, cyclodextrines, e.g. hydroxypropyl-β-cyclodextrine, sulfobutylethyl-β-cyclodextrin, β-cyclodextrin, polyethylenglycols, e.g. PEG 3000, PEG 3350, PEG 4000, PEG 6000, albumin, human serum albumin (HSA), bovine serum albumin (BSA), salts, e.g. sodium chloride, magnesium chloride, calcium chloride, chelators, e.g. EDTA as hereafter defined. Stabilizers that are particularly used in the present invention, are selected from the group consisting of sugars, polyols and amino acids. More particularly, the stabilizers are selected from the group consisting of sucrose, trehalose, sorbitol and methionine.

More preferably, the stabilizer is methionine. Methionine was used in the formulations described herein for the first time for use in ocular applications. Preclinical safety testings showed that methionine shows a good safety profile for use in ocular diseases when administered e.g. intravitreally.

Stabilizers can be present in the formulation in a concentration of about 2 mM to about 600 mM, particularly, if the stabilizer is methionine, in a concentration of about 2 mM to about 15 mM or 5 to 12 mM; more particularly in a concentration of about of 5 to 9 mM or about 7 mM.

In one preferred embodiment the stabilizer is methionine in a concentration of 7.0 mM±2.0 mM methionine (in one embodiment 7.0 mM±1.0 mM methionine; in one embodiment 7.0 mM±0.7 mM methionine). Methionine as stabilizer is especially useful as it can function in addition as scavenger agent for hydrogen peroxide which is used for sterilization of injection solutions or packaged prefilled syringes.

In some embodiments, the liquid pharmaceutical formulation of the present invention comprises an antioxidant as a second stabilizer. An “antioxidant” is a pharmaceutically acceptable excipient, which prevents oxidation of the active pharmaceutical ingredient. Antioxidants include but are not limited to chelating agents such as EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol, benzyl alcohol and n-acetyl cysteine. Antioxidants can be used in a concentration of about 0.01 to about 100 mM, particularly in a concentration of about 5 to about 50 mM and more particularly in a concentration of about 5 to about 25 mM. In particular, methionine is chosen as a second stabilizer, particularly in a concentration of about 5 to about 25 mM, more particularly in a concentration of about 10 mM.

The term “sugar” as used herein denotes a monosaccharide or an oligosaccharide. A monosaccharide is a monomeric carbohydrate which is not hydrolysable by acids, including simple sugars and their derivatives, e.g. aminosugars. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a chain. The monomeric saccharide units within an oligosaccharide can be identical or different. Depending on the number of monomeric saccharide units the oligosaccharide is a di-, tri-, tetra- penta- and so forth saccharide. In contrast to polysaccharides, the monosaccharides and oligosaccharides are water soluble. Examples of oligosaccharides include sucrose, trehalose, lactose, maltose and raffinose. In particular, sugars are selected from sucrose and trehalose, in particular sucrose.

The term “amino acid” as used herein denotes in general a pharmaceutically acceptable organic molecule possessing an amino moiety located at α-position to a carboxylic group. Examples of amino acids include arginine glycine, ornithine, lysine, histidine, glutamic acid, asparagic acid, isoleucine, leucine, alanine, phenylalanine, tyrosine, tryptophane, methionine, serine, proline, in particular methionine.

The term “polyols” as used herein denotes pharmaceutically acceptable alcohols with more than one hydroxy group. Suitable polyols comprise to but are not limited to mannitol, sorbitol, glycerine, dextran, glycerol, arabitol, propylene glycol, polyethylene glycol, and combinations thereof. Polyols can be used in a concentration of about 10 mM to about 500 mM, particularly in a concentration of about 10 to about 250 mM and more particularly in a concentration of about 200 to about 250 mM.

The term “stabilizers” also includes lyoprotectants. The term “lyoprotectant” denotes a pharmaceutical acceptable excipient, which protects the labile active ingredient (e.g. a protein) against destabilizing conditions during the lyophilisation process, subsequent storage and reconstitution. Lyoprotectants comprise but are not limited to the group consisting of sugars, polyols (such as e.g. sugar alcohols) and amino acids. In particular, lyoprotectants can be selected from the group consisting of sugars such as sucrose, trehalose, lactose, glucose, mannose, maltose, galactose, fructose, sorbose, raffinose, neuraminic acid, amino sugars such as glucosamine, galactosamine, N-methylglucosamine (“Meglumine”), polyols such as mannitol and sorbitol, and amino acids such as methionine or glycine. Lyoprotectants are generally used in a concentration of about 10 to about 600 mM, particularly in a concentration of about 10 to about 250 mM and more particularly in a concentration of about 100 to about 250 mM.

### Tonicity Agents

The pharmaceutical formulation may also contain tonicity agents. The term “tonicity agents” as used herein denotes pharmaceutically acceptable tonicity agents which are used to modulate the tonicity of the formulation. The formulation can be hypotonic, isotonic or hypertonic. Isotonicity in general relates to the osmotic pressure relative of a solution usually relative to that of human blood serum. The formulation according to the invention can be hypotonic, isotonic or hypertonic, preferably the pharmaceutical formulation is isotonic. An isotonic formulation is liquid or liquid reconstituted from a solid form, e.g. from a lyophilised form and denotes a solution having a similar tonicity as some other solution with which it is compared, such as physiologic salt solution and the blood serum. Suitable tonicity agents comprise but are not limited to sodium chloride, potassium chloride, glycerin and any component from the group of amino acids, sugars, in particular sucrose. In one embodiment of the present invention, the preferred tonicity agent is sucrose. Tonicity agents are generally used in a concentration of about 5 mM to about 1000 mM, in particular about 30 mM to about 500 mM, more particular about 120 mM to about 200 mM. Tonicity agents for isotonic formulations of the present invention are generally used in a concentration of about 50 mM to about 250 mM, in particular about 120 mM to about 200 mM. More particularly, tonicity agents for isotonic formulations are used in a concentration of 130 mM to 190 mM, and even more particularly in a concentration of about 160 mM±24 mM in case sucrose is used as tonicity agent. The tonicity agent and its concentration is chosen to enable an isotonic formulation with a target osmolality of 300±100 mOsm/kg (in particular with a target osmolality of 300±50 mOsm/kg.)

Within the stabilizers and tonicity agents there is a group of compounds which can function in both ways, i.e. they can at the same time be a stabilizer and a tonicity agent. Examples thereof can be found in the group of sugars, amino acids, polyols, cyclodextrines, polyethyleneglycols and salts. An example for a sugar which can at the same time be a stabilizer and a tonicity agent is sucrose and trehalose, in particular sucrose.

### Viscosity Reducer

The pharmaceutical formulation may also contain viscosity reducers. The term “viscosity reducers” as used herein denotes pharmaceutically acceptable ionic strength modifier which are used to reduce the viscosity of the formulation, which is important for high concentrations formulations and formulations which are foreseen to be administered intravitreally in the eye through thin needles (enabling relative fast application without the need of high pressure for the injection) in the treatment of ocular diseases. Examples of typical viscosity reducers are e.g. calcium chloride or sodium chloride.

### Adjuvants

The pharmaceutical formulation may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol, sorbic acid, and the like. Preservatives are generally used in a concentration of about 0.001 to about 2% (w/v). Preservatives comprise but are not limited to ethanol, benzyl alcohol, phenol, m-cresol, p-chlor-m-cresol, methyl or propyl parabens, benzalkonium chloride.

The pharmaceutical formulation may also contain amounts of the different above mentioned agents such as buffers, surfactants, stabilizer, ion strength modifier, in smaller amounts that do not essentially change the technical features of pharmaceutical formulation of the present invention as e.g. a viscosity of 20 mPas or less (preferably 15mPas or less), a turbidity of 30 FTU or less (preferably 25 FTU or less), an osmolality of 300±50 mOsm/kg, essentially free of visible particles.

### Use

The pharmaceutical formulation of bispecific anti-VEGF/ANG2 antibody according to the invention can be used in the prevention or treatment of ocular vascular diseases. For this purpose the pharmaceutical formulation of bispecific anti-VEGF/ANG2 antibody is provided for intravitreal administration as liquid isotonic formulation with a viscosity of 20 mPas or less (preferably 15mPas or less), a turbidity of 30 FTU or less (preferably 25 FTU or less), an osmolality of 300±50 mOsm/kg, essentially free of visible particles. The liquid isotonic pharmaceutical formulation of bispecific anti-VEGF/ANG2 antibody for this purpose can be provided in glass vials or in form of a prefilled syringe, in particular in form of a prefilled glass syringe.

For such formulations which are administered intravitreally to the eye excipients like e.g. arginine should be avoided as the toxicity of arginine as carrier for tissue plasminogen activator (t-PA), to the retina and retinal pigment epithelium has been described (see e.g. Benner J D, Morse L S, Toth C A et al Arch Ophthalmol 19911091731-1736.1736; Johnson M W, Olsen K R. Hernandez E. et al, Arch Ophthalmool 1990108259-263.263, Irvine W D, Johnson M W, Heinandez E. et al., Arch Ophthalmol 1991109718-722.722, Johnson M W, Olsen K R. Heinandez E., Retina 199111250-258.258). Therefore, the liquid pharmaceutical formulation of the present invention is essentially free of arginine (which means the formulation comprises no arginine or amounts of arginine below a concentration/level where they can be toxic (via their function as carrier for tissue plasminogen activator (t-PA)) or does not comprise arginine. A low viscosity is also essential in order to enable a commercial-scale production process (up-concentration by ultrafiltration) and to ensure an easy and convenient intravitreal injection (injection (gliding) forces of less than 20 N, in particular less than 15N with an injection speed of 50 mm/min). It was demonstrated that the liquid pharmaceutical formulation of the present invention with a viscosity of less than 15 mPas were able to be injected through a 30G injection needle with 5s injection time with an injection force of less than 5 N. For this purpose, the pharmaceutical formulation of bispecific anti-VEGF/ANG2 antibody is as liquid isotonic formulation with a viscosity of 15 mPas or less, a turbidity of 25 FTU or less, an osmolality of 300±50 mOsm/kg, essentially free of visible particles. To avoid any visible particles, the liquid pharmaceutical formulation of the present invention is essentially free of calcium chloride (which means the formulation comprises no calcium chloride or comprises amounts of calcium chloride below a concentration/level where they can contribute to the formation of visible particles, so that the formulation remains/is essentially free of visible particles) or does not comprise calcium chloride.

The terms “ocular vascular disease” and “vascular eye disease” are used interchangeable herein and include, but are not limited to intraocular neovascular syndromes such as diabetic retinopathy, diabetic macular edema, retinopathy of prematurity, neovascular glaucoma, retinal vein occlusions, central retinal vein occlusions, macular degeneration, age-related macular degeneration, retinitis pigmentosa, retinal angiomatous proliferation, macular telangectasia, ischemic retinopathy, iris neovascularization, intraocular neovascularization, corneal neovascularization, retinal neovascularization, choroidal neovascularization, and retinal degeneration. (Garner, A., Vascular diseases, In: Pathobiology of ocular disease, A dynamic approach, Garner, A., and Klintworth, G. K., (eds.), 2nd edition, Marcel Dekker, New York (1994), pp. 1625-1710). As used herein, ocular vascular disorder refers to any pathological conditions characterized by altered or unregulated proliferation and invasion of new blood vessels into the structures of ocular tissues such as the retina or cornea. In one embodiment the ocular vascular disease is selected from the group consisting of: wet age-related macular degeneration (wet AMD) (also called neovascular age-related macular degeneration (nAMD)), diabetic macular edema (DME), diabetic retinopathy (DR), non-proliferative diabetic retinopathy (NPDR), proliferative diabetic retinopathy (PDR), cystoid macular edema (CME), vasculitis (e.g. central retinal vein occlusion), retinal vein occlusion (RVO), central retinal vein occlusion (CRVO), papilledema, retinitis, conjunctivitis, uveitis, choroiditis, multifocal choroiditis, ocular histoplasmosis, blepharitis, dry eye (Sjögren's disease) and other ophthalmic diseases wherein the eye disease or disorder is associated with ocular neovascularization, vascular leakage, and/or retinal edema, in particular wet age-related macular degeneration (wet AMD) (also called neovascular age-related macular degeneration (nAMD)), diabetic macular edema (DME), diabetic retinopathy (DR), non-proliferative diabetic retinopathy (NPDR), proliferative diabetic retinopathy (PDR), cystoid macular edema (CME), vasculitis (e.g. central retinal vein occlusion), retinal vein occlusion (RVO), central retinal vein occlusion (CRVO). So the anti-VEGF/ANG2 bispecific antibodies for use and the methods described herein are useful in the prevention and treatment of wet AMD (also called neovascular age-related macular degeneration (nAMD)), DME, DR, NPDR, PDR, also preferably wet AMD, DME, and RVO, also preferably wet AMD wet AMD. In some embodiments, the ocular vascular disease is selected from the group consisting wet age-related macular degeneration (wet AMD)), diabetic macular edema (DME), retinal vein occlusions (RVO), diabetic retinopathy (DR). and retinopathy of prematurity (ROP),

Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, and corneal graph rejection.

Diseases associated with retinal/choroidal neovascularization include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, retinitis pigmentosa, retina edema (including macular edema), Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications. Other diseases include, but are not limited to, diseases associated with rubeosis (neovascularization of the angle) and diseases caused by the abnormal proliferation of fibrovascular or fibrous tissue including all forms of proliferative vitreoretinopathy.

Retinopathy of prematurity (ROP) is a disease of the eye that affects prematurely born babies. It is thought to be caused by disorganized growth of retinal blood vessels which may result in scarring and retinal detachment. ROP can be mild and may resolve spontaneously, but may lead to blindness in serious cases. As such, all preterm babies are at risk for ROP, and very low birth weight is an additional risk factor. Both oxygen toxicity and relative hypoxia can contribute to the development of ROP.

Macular degeneration is a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, or to recognize faces. According to the American Academy of Ophthalmology, it is the leading cause of central vision loss (blindness) in the United States today for those over the age of fifty years. Although some macular dystrophies that affect younger individuals are sometimes referred to as macular degeneration, the term generally refers to age-related macular degeneration (AMD or ARMD).

“Age-related macular degeneration (AMD)”, as used herein, refers to a serious eye condition when the small central portion of the retina, known as the macula, deteriorates. The wet form of AMD (wet AMD (wAMD), also called neovascular AMD (nAMD)), a form of advanced AMD is characterized by the growth of abnormal blood vessels from the choroid underneath the macula. This is called choroidal neovascularization. These blood vessels leak blood and fluid into the retina, causing distortion of vision that makes straight lines look wavy, as well as blind spots and loss of central vision. These abnormal blood vessels eventually scar, leading to permanent loss of central vision. The symptoms of AMD include dark, blurry areas in the center of vision; and diminished or changed color perception. AMD can be detected in a routine eye exam. One of the most common early signs of macular degeneration is the presence of drusen tiny yellow deposits under the retina or pigment clumping.

Retinitis pigmentosa (RP) is a group of genetic eye conditions. In the progression of symptoms for RP, night blindness generally precedes tunnel vision by years or even decades. Many people with RP do not become legally blind until their 40s or 50s and retain some sight all their life. Others go completely blind from RP, in some cases as early as childhood. Progression of RP is different in each case. RP is a type of hereditary retinal dystrophy, a group of inherited disorders in which abnormalities of the photoreceptors (rods and cones) or the retinal pigment epithelium (RPE) of the retina lead to progressive visual loss. Affected individuals first experience defective dark adaptation or nyctalopia (night blindness), followed by reduction of the peripheral visual field (known as tunnel vision) and, sometimes, loss of central vision late in the course of the disease.

Macular edema occurs when fluid and protein deposits collect on or under the macula of the eye, a yellow central area of the retina, causing it to thicken and swell. The swelling may distort a person's central vision, as the macula is near the center of the retina at the back of the eyeball. This area holds tightly packed cones that provide sharp, clear central vision to enable a person to see form, color, and detail that is directly in the line of sight. Cystoid macular edema is a type of macular edema that includes cyst formation.

“Diabetic Macular Edema” (DME), as used herein, refers to a serious eye condition that affects people with diabetes (type 1 or 2). Macular edema occurs when blood vessels in the retina leak into the macula and fluid and protein deposits collect on or under the macula of the eye (a yellow central area of the retina) and causes it to thicken and swell (edema). The swelling may distort a person's central vision, as the macula is near the center of the retina at the back of the eyeball. The primary symptoms of DME include, but are not limited to, blurry vision, floaters, loss of contrast, double vision, and eventual loss of vision. The pathology of DME is characterized by breakdown of the blood-retinal barrier, normally preventing water movement in the retina, thus allowing fluid to accumulate in the retinal tissue, and presence of retinal thickening. DME is presently diagnosed during an eye examination consisting of a visual acuity test, which determines the smallest letters a person can read on a standardized chart, a dilated eye exam to check for signs of the disease, imaging tests such as optical coherence tomography (OCT) or fluorescein angiography (FA) and tonometry, an instrument that measures pressure inside the eye. The following studies are also performed to determine treatment: optical coherence tomography (OCT), fluorescein angiography, and color stereo fundus photography. DME can be broadly characterized into two main categories—Focal and Diffuse. Focal DME is characterized by specific areas of separate and distinct leakage in the macula with sufficient macular blood flow. Diffuse DME results from leakage of the entire capillary bed surrounding the macula, resulting from a breakdown of the inner blood-retina barrier of the eye. In addition to Focal and Diffuse, DME is also categorized based on clinical exam findings into clinically significant macular edema (CSME), non-CSME and CSME with central involvement (CSME-CI), which involves the fovea. The present invention includes methods to treat the above-mentioned categories of DME.

In one embodiment of the invention the ocular vascular disease is selected from the group consisting of: wet age-related macular degeneration (wet AMD), diabetic macular edema (DME), diabetic retinopathy (DR), non-proliferative diabetic retinopathy (NPDR), proliferative diabetic retinopathy (PDR), vasculitis (e.g. retinal vein occlusion (RVO) and central retinal vein occlusion (CRVO.

In one embodiment the ocular vascular disease is selected from the group consisting of diabetic retinopathy (DR), diabetic macular edema (DME), retinal vein occlusion (RVO), central retinal vein occlusion (CRVO), macular degeneration, wet age-related macular degeneration (wet AMD), retinopathy of prematurity (ROP), neovascular glaucoma, retinitis pigmentosa (RP), retinal angiomatous proliferation, macular telangiectasia, ischemic retinopathy, iris neovascularization, intraocular neovascularization, corneal neovascularization, retinal neovascularization, choroidal neovascularization, and retinal degeneration, in particular from the group consisting of diabetic retinopathy (DR), diabetic macular edema (DME), retinal vein occlusion (RVO), central retinal vein occlusion (CRVO), wet age-related macular degeneration (wet AMD).

In one embodiment of the invention the ocular vascular disease is diabetic retinopathy (DR).

In one embodiment of the invention the ocular vascular disease is diabetic macular edema (DME).

In one embodiment of the invention the ocular vascular disease is retinal vein occlusion (RVO).

In one embodiment of the invention the ocular vascular disease is central retinal vein occlusion (CRVO).

In one embodiment of the invention the ocular vascular disease is retinopathy of prematurity (ROP).

In one embodiment of the invention the ocular vascular disease is macular degeneration.

In one embodiment of the invention the ocular vascular disease is age-related macular degeneration (AMD).

In one embodiment of the invention the ocular vascular disease is wet age-related macular degeneration (wAMD).

In one embodiment of the invention the ocular vascular disease is choroidal neovascularization.

### Administration

The liquid pharmaceutical formulation according to the invention can be administered by intravitreal (IVT) means such as those known in the pharmaceutical art (e.g an appropriate syringe). For the intravitreal injection typically injection volumes are 50 to 100 μL. The intravitreal injection is performed by use of a disposable syringe and an injection needle of 30G (25G to 30G) or a pre-filled syringe with an appropriate injection needle. The liquid formulation can be withdrawn from the vial containing the formulation by use of a filter needle with pore size of 5 μm. Intravitreal injection technique is described e.g. in D. Yorston, Community Eye Health. 2014; 27(87): 47.

For this purpose, the pharmaceutical formulation of bispecific anti-VEGF/ANG2 antibody is as liquid isotonic formulation with a viscosity of 15mPas or less, a turbidity of 25 FTU or less, an osmolality of 300±50 mOsm/kg, essentially free of visible particles.

The formulations to be used for in vivo administration must be sterile. This is readily accomplished by filtration through sterile filtration membranes and aseptic manufacturing practice.

### Method for the Preparation of the Formulation

The pharmaceutical formulation according to the invention can be prepared by methods or processes known in the art, e.g. ultrafiltration-diafiltration, dialysis, addition and mixing, lyophilisation, reconstitution, and combinations thereof. Examples of preparations of formulations according to the invention can be found herein after.

In one embodiment of the invention the pharmaceutical formulation can be prepared by the following manufacturing method or process comprising the steps of:

1. Buffer exchange against a diafiltration buffer containing the histidine-acetate buffer or the histidine-acetate buffer and sodium chloride, or histidine-acetate buffer, sodium chloride and methionine, or histidine-acetate buffer, sodium chloride, methionine and sucrose by ultra-filtration and diafiltration using a semipermeable membrane with a MWCO (molecular weight cut-off) between 5 and 80 kD (30 to 50 kD) (30 kD). Typically, the ratio between diafiltration buffer and bulk solution is 5 to 20 (5-10).

2. Alternatively to 1, buffer-exchange can be achieved by dialysis using a dialysis buffer containing the histidine-acetate buffer or the histidine-acetate buffer and sodium chloride, or histidine-acetate buffer, sodium chloride and methionine, or histidine-acetate buffer, sodium chloride, methionine and sucrose and dialysis membrane with a MWCO between 5 and 80 kD (30 to 50 kD) (30 kD). Typically, the ratio between dialysis buffer and bulk solution is 5 to 20 (5-10).

3. The buffer exchanged bulk solution is concentrated by ultrafiltration using a diafiltration membrane with a MWCO (molecular weight cut-off) between 5 and 80 kD (30 to 50 kD) (30 kD) to a protein concentration of more than 120 mg/mL (120 to 160 mg/mL) (120 to 200 mg/mL).

4. The final composition of the pharmaceutical formulation is adjusted by addition of stock solutions of the respective excipients or by an appropriate conditioning buffer. The solution is homogenized by mixing.

Furthermore, the manufacturing method or process can include the following steps

5. The final formulated solution can be stored frozen at a temperature below −20° C. (below −40° C.).

6. Before filling in the final primary container the solution is thawed

7. Several container or batches of the pharmaceutical formulation are mixed and homogenized by stirring

8. The homogenized pharmaceutical formulation is filtered through several (at least two) sterilizing grade filters with a pore size of at least 0.2 or 0.22 μm.

9. The sterile-filtered solution is filled under aseptic conditions into sterile vials or pre-fillable syringes and closed with elastomeric stopper, respectively plunger stopper and tip caps.

10. The filled primary containers are inspected for defects and visible particles

11. Pre-filled syringes are assembled with respective device components, packaged into sterile barrier system and sterilized at the outer surface.

12. Vials and sterilized syringes are packaged in the final secondary packaging

The pharmaceutical formulations according to the invention can also be in a lyophilized form or in a liquid form reconstituted from the lyophilized form. The “lyophilized form” is manufactured by freeze-drying methods known in the art. The lyophilizate usually has a residual moisture content of about 0.1 to 5% (w/w) and is present as a powder or a physically stable cake. The “reconstituted form” can be obtained from the lyophilizate by a fast dissolution after addition of reconstitution medium. Suitable reconstitution media comprise but are not limited to water for injection (WFI), bacteriostatic water for injection (BWFI), sodium chloride solutions (e.g. 0.9% (w/v) NaCl), and glucose solutions (e.g. 5% (w/v) glucose).

### Production of the Antibodies

Anti-VEGF/ANG2 antibodies that are particularly useful for the invention are produced by recombinant means. Methods for recombinant production are widely known in the state of the art and comprise protein expression in prokaryotic and eukaryotic cells with subsequent isolation of the antibody and usually purification to a pharmaceutically acceptable purity. For the expression of the antibodies as aforementioned in a host cell, nucleic acids encoding the respective modified light and heavy chains are inserted into expression vectors by standard methods. Expression is performed in appropriate prokaryotic or eukaryotic host cells like CHO cells, NSO cells, SP2/0 cells, HEK293 cells, COS cells, PER.C6 cells, yeast, or E. coli cells, and the antibody is recovered from the cells (supernatant or cells after lysis). General methods for recombinant production of antibodies are well-known in the state of the art and described, for example, in the review articles of Makrides, S. C., Protein Expr. Purif. 17 (1999) 183-202; Geisse, S., et al, Protein Expr. Purif. 8 (1996) 271-282; Kaufman, R. J., Mol. Biotechnol 16 (2000) 151-160; Werner, R. G., Drug Res. 48 (1998) 870-880. A method for the preparation of an antibody useful in the invention, comprises the steps of a) transforming a host cell with vectors comprising nucleic acid molecules encoding said antibody; b) culturing the host cell under conditions that allow synthesis of said antibody molecule; and c) recovering said antibody molecule from said culture.

The antibodies are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies is readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant monoclonal antibodies in the host cells.

Amino acid sequence variants (or mutants) of the bispecific antibody are prepared by introducing appropriate nucleotide changes into the antibody DNA, or by nucleotide synthesis. Such modifications can be performed, however, only in a very limited range. For example, the modifications do not alter the above mentioned antibody characteristics such as the IgG isotype and antigen binding, but may improve the yield of the recombinant production, protein stability or facilitate the purification.

The term “host cell” as used in the current application denotes any kind of cellular system which can be engineered to generate the antibodies comprised in the formulation of the current invention. In one embodiment HEK293 cells and CHO cells are used as host cells.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included.

Expression in NS0 cells is described by, e.g., Barnes, L. M., et al, Cytotechnology 32 (2000) 109-123; Barnes, L. M., et al, Biotech. Bioeng. 73 (2001) 261-270. Transient expression is described by, e.g., Durocher, Y., et al, Nucl. Acids. Res. 30 (2002) E9. Cloning of variable domains is described by Orlandi, R., et al, Proc. Natl. Acad. Sci. USA 86 (1989) 3833-3837; Carter, P., et al, Proc. Natl. Acad. Sci. USA 89 (1992) 4285-4289; and Norderhaug, L., et al, J. Immunol. Methods 204 (1997) 77-87. A preferred transient expression system (HEK 293) is described by Schlaeger, E.-J., and Christensen, K., in Cytotechnology 30 (1999) 71-83 and by Schlaeger, E.-J., in J. Immunol. Methods 194 (1996) 191-199.

The control sequences that are suitable for prokaryotes, for example, include a promoter, optionally an operator sequence, and a ribosome binding site. Eukaryotic cells are known to utilize promoters, enhancers and polyadenylation signals.

A nucleic acid is “operably linked” when it is placed in a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

Purification of antibodies is performed in order to eliminate cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al, ed. Current Protocols in Molecular Biology, Greene Publishing and Wiley Interscience, New York (1987). Different methods are well established and widespread used for protein purification, such as affinity chromatography with microbial proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A., Appl. Biochem. Biotech. 75 (1998) 93-102).

### Amino Acid Sequences Disclosed in the Application:

| SEQ | |
|---|---|
| ID | |
| NO: | Description |
| 1 | heavy chain variable domain CDR3 of <VEGF> |
| 2 | heavy chain variable domain CDR2 of <VEGF> |
| 3 | heavy chain variable domain CDR1 of <VEGF> |
| 4 | light chain variable domain CDR3 of <VEGF> |
| 5 | light chain variable domain CDR2 of <VEGF> |
| 6 | light chain variable domain CDR1 of <VEGF> |
| 7 | heavy chain variable domain CDR3 of <ANG-2> |
| 8 | heavy chain variable domain CDR2 of <ANG-2> |
| 9 | heavy chain variable domain CDR1 of <ANG-2> |
| 10 | light chain variable domain CDR3 of <ANG-2> |
| 11 | light chain variable domain CDR2 of <ANG-2> |
| 12 | light chain variable domain CDR1 of <ANG-2> |
| 13 | heavy chain variable domain (VH) of <VEGF> |
| 14 | light chain variable domain (VL) of <VEGF> |
| 15 | heavy chain variable domain (VH) of <ANG-2> |
| 16 | light chain variable domain (VL) of <ANG-2> |
| 17 | Bispecific anti-VEGF/ANG2 antibody: CrossMAb VEGFang2- |
| | 0016-<VEGF> light chain |
| 18 | Bispecific anti-VEGF/ANG2 antibody: CrossMAb VEGFang2- |
| | 0016-<ANG2> light chain |
| 19 | Bispecific anti-VEGF/ANG2 antibody: CrossMAb VEGFang2- |
| | 0016-<VEGF> heavy chain |
| 20 | Bispecific anti-VEGF/ANG2 antibody: CrossMAb VEGFang2- |
| | 0016-<ANG2> heavy chain |
| 21 | Exemplary kappa constant light chain region |
| 22 | Exemplary lambda constant light chain region |
| 23 | Exemplary constant heavy chain region of human IgG1 subclass |
| 24 | Human angiopoietin-2 (ANG-2) with leader and His-tag |
| 25 | human vascular endothelial growth factor (VEGF) |

In the following embodiments of the invention are listed:
1. A liquid pharmaceutical formulation comprising:
   20 to 150 mg/ml of a bispecific anti-VEGF/ANG2 antibody comprising a constant heavy chain region of human IgG1 subclass (in one embodiment 30 mg/ml±4.5 mg/ml or 120 mg/ml±18 mg/ml),
   15 to 35 mM of sodium chloride (in one embodiment 25 mM±5 mM; in one embodiment 25 mM±3.75 mM of sodium chloride; in particular, 25 mM±2.5 mM of sodium chloride),
   15 to 25 mM of a histidine acetate buffer (in one embodiment 20 mM±3 mM of a histidine acetate buffer; in one embodiment 20 mM±2 mM of a histidine acetate buffer),
   at a pH of 5.5±0.5 (in one embodiment at a pH of 5.5±0.3; in particular, at a pH of 5.5±0.2);
wherein the bispecific anti-VEGF/ANG2 antibody is bivalent and comprises a first antigen-binding site that specifically binds to human VEGF and a second antigen-binding site that specifically binds to human ANG-2, wherein
   i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
   ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14,
   and wherein
   iii) the bispecific antibody comprises a constant heavy chain region of human IgG1 subclass comprising the mutations I253A, H310A, and H435A and the mutations L234A, L235A and P329G (numberings according to EU Index of Kabat.
2. The pharmaceutical formulation according to embodiment 1, wherein the bispecific anti-VEGF/ANG2 antibody is bivalent and comprises a first antigen-binding site that specifically binds to human VEGF and a second antigen-binding site that specifically binds to human ANG-2, wherein
   i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
   ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.
3. The pharmaceutical formulation according to embodiment 2, wherein the bispecific anti-VEGF/ANG2 antibody is bivalent and comprises a first antigen-binding site that specifically binds to human VEGF and a second antigen-binding site that specifically binds to human ANG-2, wherein
   wherein
   iv) in the constant heavy chain region a S354C and T366W mutations are comprised in one CH3 domain and Y349C, T366S, L368A and Y407V mutations are comprised the other CH3 domain (numberings according to EU Index of Kabat).
4. The pharmaceutical formulation according to any one of embodiments 1 to 3, wherein the bispecific anti-VEGF/ANG2 antibody is bivalent and comprises the amino acid sequences of SEQ ID NO: 17, of SEQ ID NO: 18, of SEQ ID NO: 19, and of SEQ ID NO: 20.
5. The pharmaceutical formulation according to any one of embodiments 1 to 3, wherein the bispecific anti-VEGF/ANG2 antibody is faricimab.
6. The pharmaceutical formulation according to embodiment 1, wherein the formulation comprises
   120 mg/ml±18 mg/ml of the bispecific anti-VEGF/ANG2 antibody (in particular 120 mg/ml±12 mg/ml of the bispecific anti-VEGF/ANG2 antibody).
7. The pharmaceutical formulation according to any one of claims 1 to 7 for intravitreal administration.
8. The pharmaceutical formulation according to any one of embodiments 1 to 6, wherein the formulation is essentially free of visible particles.
9. The pharmaceutical formulation according to any one of embodiments 1 to 8, wherein the formulation further comprises
   1 to 20 mM of at least one stabilizer (in one embodiment selected from sugars, polyols and amino acids).
10. The pharmaceutical formulation according to any one of embodiments 1 to 8, wherein the formulation further comprises
   7.0 mM±2.0 mM methionine (in one embodiment 7.0 mM±1.0 mM methionine; in one embodiment 7.0 mM±0.7 mM methionine).
11. The pharmaceutical formulation according to any one of embodiments 1 to 10, wherein the formulation further comprises
   0.01-0.07% of a surfactant (in one embodiment selected from polysorbate 20, polysorbate 80, or poloxamer).
12. The pharmaceutical formulation according to any one of embodiments 1 to 10, wherein the formulation further comprises
   0.04% (w/v)±0.02 (w/v) polysorbate 20 (in one embodiment 0.03% (w/v) to 0.07% (w/v)); in one embodiment 0.04% (w/v)±0.01 (w/v); in one embodiment about 0.04% (w/v))
13. The pharmaceutical formulation according to any one of embodiments 1 to 12, wherein the formulation further comprises
   50-250 mM of a tonicity agent (in one embodiment selected from the tonicity agent is selected from sucrose, trehalose, and sorbitol).
14. The pharmaceutical formulation according to any one of embodiments 1 to 12, wherein the formulation further comprises
   160 mM±24 mM sucrose (in one embodiment 160 mM±16 mM; in one embodiment about 160 mM).
15. The pharmaceutical formulation according to any one of embodiments 1 to 14, wherein the formulation has a viscosity of 20 mPas or less (in one embodiment 17 mPas or less; in one embodiment 16 mPas or less, in one embodiment about 15 mPas or less; in one embodiment 15 mPas or less).
16. The pharmaceutical formulation according to any one of embodiments 1 to 15, wherein the formulation has a turbidity of 30 FTU or less (in one embodiment 27 FTU or less; in one embodiment 26 FTU or less; in one embodiment about 25 FTU or less; in one embodiment 25 FTU or less).
17. The pharmaceutical formulation according to any one of embodiments 1 to 16, wherein the formulation has an ionic strength between 20 and 50 (in one embodiment an ionic strength between 30 and 50 (in one embodiment an ionic strength between 30 and 45; in one embodiment an ionic strength of 30±10).
18. The pharmaceutical formulation according to any one of embodiments 1 to 17 for intravitreal administration wherein the formulation is essentially free of calcium chloride (or does not comprise calcium chloride).
19. The pharmaceutical formulation according to any one of embodiments 1 to 17 for intravitreal administration wherein the formulation is essentially free of arginine (or does not comprise arginine).
20. The pharmaceutical formulation according to any one of embodiments 1 to 17 for intravitreal administration wherein the formulation is essentially free of arginine and calcium chloride (or does not comprise arginine and calcium chloride).
21. The pharmaceutical formulation according to any one of embodiments 1 to 5 and 7 to 20, wherein the formulation comprises or consists (at least) of the following components
   20 to 150 mg/ml of the bispecific anti-VEGF/ANG2 antibody (in one embodiment 30 mg/ml±4.5 mg/ml or 120 mg/ml±18 mg/ml, in particular 120 mg/ml±12 mg/ml);
   15 to 35 mM of sodium chloride (in one embodiment 25 mM±5 mM; in one embodiment 25 mM±3.75 mM of sodium chloride; in particular, 25 mM±2.5 mM of sodium chloride);
   15 to 25 mM of histidine acetate buffer (in one embodiment 20 mM±3 mM of a histidine acetate buffer; in one embodiment 20 mM±2 mM of a histidine acetate buffer);
   7.0 mM±2.0 mM methionine (in one embodiment 7.0 mM±1.0 mM methionine; in one embodiment 7.0 mM±0.7 mM methionine)
   0.03% (w/v) to 0.07% (w/v) polysorbate 20 (in one embodiment 0.04% (w/v)±0.02 (w/v); in one embodiment 0.04% (w/v)±0.01 (w/v); in one embodiment about 0.04% (w/v));
   160 mM±24 mM sucrose (in one embodiment 160 mM±16 mM; in one embodiment about 160 mM);
   water (for (ophthalmologic) injections);
at a pH of 5.5±0.5 (in one embodiment at a pH of 5.5±0.3; in particular, at a pH of 5.5±0.2);
22. The pharmaceutical formulation according to any one of embodiments 1 to 21, wherein formulation is a stable formulation.
23. The pharmaceutical formulation according to any one of embodiments 1 to 21, wherein the high molecular weight species (HMW) content of the bispecific antibody in the pharmaceutical formulation is below 10% after 8 weeks at 25° C. or after 52 weeks at 25° C. (in one embodiment below 5%).
24. The pharmaceutical formulation according to any one of embodiments 1 to 21, wherein the main peak of the bispecific antibody in the pharmaceutical formulation is more than 50% and the high molecular weight species (HMW) content of the bispecific antibody in the pharmaceutical formulation is below 10% after 2 years at 2-8° C. (or after 3 years at 2-8° C.) (the main peak of the bispecific antibody in the pharmaceutical formulation is more than 55% and the high molecular weight species (HMW) content of the bispecific antibody in the pharmaceutical formulation is below 7% after 2 years at 2-8° C. (or after 3 years at 2-8° C.)).
25. The pharmaceutical formulation according to any one of embodiments 1 to 24, wherein the osmolality of the formulation is 300±100 mOsm/kg (in one embodiment 300±50 mOsm/kg).
26. The pharmaceutical formulation according to any one of embodiments 1 to 25 for use in the treatment of an ocular vascular disease.
27. The pharmaceutical formulation for use according to embodiment 26, wherein the ocular vascular diseases is selected from the group consisting of diabetic retinopathy (DR), diabetic macular edema (DME), retinal vein occlusion (RVO), central retinal vein occlusion (CRVO), macular degeneration, wet age-related macular degeneration (wet AMD), retinopathy of prematurity (ROP), neovascular glaucoma, retinitis pigmentosa (RP), retinal angiomatous proliferation, macular telangiectasia, ischemic retinopathy, iris neovascularization, intraocular neovascularization, corneal neovascularization, retinal neovascularization, choroidal neovascularization, and retinal degeneration, in particular from the group consisting of diabetic retinopathy (DR), diabetic macular edema (DME), retinal vein occlusion (RVO), central retinal vein occlusion (CRVO), wet age-related macular degeneration (wet AMD).
28. The pharmaceutical formulation for use according to embodiment 26, wherein the ocular vascular disease is diabetic retinopathy.
29. The pharmaceutical formulation for use according to embodiment 26, wherein the ocular vascular disease is diabetic macular edema (DME).
30. The pharmaceutical formulation for use according to embodiment 26, wherein the ocular vascular disease is retinal vein occlusion (RVO).
31. The pharmaceutical formulation for use according to embodiment 26, wherein the retinal vein occlusion is central retinal vein occlusion (CRVO).
32. The pharmaceutical formulation for use according to embodiment 26, wherein the ocular vascular disease is macular degeneration.
33. The pharmaceutical formulation for use according to embodiment 26, wherein the macular degeneration is age-related macular degeneration (AMD).
34. The pharmaceutical formulation for use according to embodiment 26, wherein the macular degeneration is wet age-related macular degeneration (wAMD) (also called neovascular age-related macular degeneration (nAMD).
35. The pharmaceutical formulation for use according to embodiment 26, wherein the ocular vascular disease is choroidal neovascularization.
36. A method for the preparation of the pharmaceutical formulation of any one of embodiments 1 to 25
   the method comprising the steps of
      buffer exchange of the bispecific antibody bulk solution a) against a diafiltration buffer by ultra-filtration and diafiltration or b) by dialysis using a dialysis buffer, the buffers containing a histidine-acetate buffer or a histidine-acetate buffer and sodium chloride, or a histidine-acetate buffer, sodium chloride and methionine, or a histidine-acetate buffer, sodium chloride, methionine and sucrose
      concentration of the buffer exchanged bulk solution by ultrafiltration
      adjustment of the final composition of the pharmaceutical formulation by addition of stock solutions of the respective excipients or by an appropriate conditioning buffer and homogenization of the liquid pharmaceutical formulation is homogenized by mixing.
37. A vial comprising the pharmaceutical formulation of any one of embodiments 1 to 25.
38. A prefilled syringe comprising the pharmaceutical formulation of any one of embodiments 1 to 25.
39. A lyophilised form of the liquid pharmaceutical formulation of any one of embodiments 1 to 25.

### EXAMPLES

Liquid drug product pharmaceutical formulations for intravitreal (IVT) administration according to the invention were developed as follows.

### Example 1: Materials and Methods

The bispecific anti-VEGF/ANG2 antibody CrossMAb VEGFang2-0016 (faricimab) prepared and purified as described in WO2014/009465 was provided for further experimentation initially at a concentration of approximately 130 to 140 mg/mL in a 20 mM histidine-HCl buffer at pH 5.5.

A summary of materials (including supplier) used during the preparation of the formulations and their primary packaging is given in table 1 and table 2

TABLE 1
Chemicals used for formulations

| Chemical | Supplier |
|---|---|
| L-Histidine free Base | Ajinomoto |
| L-Histidine-HCl Monohydrate | Ajinomoto |
| Sodium-Acetate-Trihydrate | Merck |
| Glacial Acetic Acid | Wacker Chemie |
| Sodium Chloride | Merck |
| Calcium chloride Dihydrate | Applichem |
| (CaCl₂) | |
| Sucrose | FPS |
| Methionine | Ajinomoto |
| Polysorbate 20 | Croda |
| Poloxamer | BASF |

TABLE 2
Primary Packaging

| Designation | Supplier |
|---|---|
| 6 ml vials, colorless, 20 mm | Schott |
| teflonized serum-stoppers D777-1, 20 mm | Daikyo |
| Aluminum caps with PP-cap, 20 mm | Helvoet (Datwyler) |
| 2 ml vials, colorless, 13 mm | Schott |
| teflonized serum-stoppers D777-1, 13 mm | Daikyo |
| Aluminum caps with PP-cap, 13 mm | Helvoet (Datwyler) |
| 1.0 mL prefillable syringe (PFS) | Gerresheimer Buende |
| 1.0 mL PFS plunger stopper | West |
| 0.5 mL prefillable syringe (PFS) | Nuova Ompi |
| 0.5 mL PFS plunger stopper | West |

### Container Closure System

Colorless 2-mL or 6-mL glass vial (type 1 glass) closed by means of a rubber stopper (D 777-1, 13 mm) and an aluminium overseal with flip-off cap.

Colorless 1.0-mL pre-fillable syringe (type 1 glass) with luer cone closed by means of Gerresheimer Buende TELC tip cap and West 4023/50 plunger stopper.

Colorless 0.5-mL pre-fillable syringe (type 1 glass) with luer cone closed by means of Vetter OVS tip cap and West 4023/50 plunger stopper. The Vetter OVS Tip cap consists of the West 4023/50 elastomer.

### Size Exclusion Chromatography (SE-HPLC)

Size Exclusion Chromatography (SEC) was used to detect soluble high molecular weight species (aggregates) and low molecular weight hydrolysis products (LMW) in the formulations. The method was performed with a TSK-Gel® G3000SWXL, 7.8×300 mm, 5 μm (Tosoh Bioscience, Cat. no. 08541) or BioSuite 250, 7.8×300 mm, or 5 μm (Waters, Cat. no. 186002165). Intact monomer, aggregates and fragments were separated by an isocratic elution profile, using 0.2 M Potassium phosphate, 0.25 M KCl, pH 7.0 as mobile phase, and were detected at a wavelength of 280 nm.

### Ion Exchange Chromatography (IE-HPLC))

Ion Exchange Chromatography (IEC) was performed to detect chemical degradation products altering the net charge of the test antibody in the formulations. The method was performed with a YMC BioPro SP-F, 100×4.6 mm, 5 μm column (YMC, catalogue number SF00S05-1046WP). 20 mM BES (N,N-bis [2-hydroxyethyl]-2-aminoethanesulfonic acid), pH 6.8 was used as eluent A and 20 mM BES, 488 mM NaCl, pH 6.8 as eluent B, respectively, at a flow rate of 0.8 ml/min. The samples were dilute with eluent A to 3 mg/mL before injecting onto the column.

### Gradient Program:

| Time | % Mobile | % Mobile |
|---|---|---|
| (min) | Phase A | Phase B |
| 0 | 98 | 2 |
| 5 | 98 | 2 |
| 35 | 85 | 15 |
| 35.1 | 0 | 100 |
| 40 | 0 | 100 |
| 40.1 | 98 | 2 |
| 50 | 98 | 2 |

### Turbidity (in FTU (=Formazine Turbidity Unit))

The turbidity of the formulation samples was measured on a Hach 2100 AN turbidimeter according to Ph. Eur. 2.2.1 (Clarity and degree of opalescence of Liquids). A sample volume of approximately 2 mL sample solution is transferred into a 11 mm inner diameter glass cuvette and m. The glass cuvette is placed into the turbidimeter and the turbidity is measured against a calibration curve of the reference suspensions 1 FTU, 3 FTU, 10 FTU, 20 FTU and 100 FTU.

Viscosity (in mPa)

The viscosity of the formulation samples was measured on an Anton Paar Physica MCR 301 rotational rheometer with a 25 mm-0.5° cone at a shear rate of 1000 s⁻¹ and a temperature of 20° C.

### Visible Particles

The vial samples were visually inspected on a Seidenader inspection machine V90-T with help of a 2× magnifier lens. The illuminating light sources L1, L2 and L3 were adjusted to setting 5. The vial samples were inspected during a rotational movement for the presence of particles.

Protein Concentration (in mg/ml).

The protein concentration of the formulation samples was measured by ultraviolet (UV) light absorption on an UV/Vis Photometer Lambda™ 35 from Perkin Elmer. The formulation samples were diluted with a 20 mM L-histidine-acetate buffer solution pH 5.5 to a protein concentration of approximately 0.5 mg/mL and filled into a measurement cuvette with a thickness of 1 cm. The UV absorption of the measurement cuvette was measured at wavelengths at 280 and 320 nm.

The protein concentration was calculated from the measured UV light absorptions at 280 (A280) and 320 nm (A320), the extinction coefficient (E) of 1.70 mL/(mg×cm), the thickness (d) of 1 cm and dilution factor (DF) corresponding to the actual dilution according to the following equation:

$$Protein concentration in mg / mL = \frac{( A 280 - A 320 )}{( E \times d )} \times DF$$

### Osmolality

The osmolality of the formulation samples was measured on an Osmomat® 030 3P osmometer from Gonotec® according to the principle of freezing point depression.

pH

The pH of the formulations samples was determined by potentiometry with a glass electrode.

### Example 2: PH/Buffer Screen I

### Setup

The scope of the pH/buffer screen was to select the optimal pH and buffer for the commercial formulation of anti-VEGF/ANG2 antibody and to select a formulation with a low viscosity, a reduced turbidity and a good stability behavior resulting in a low formation of soluble aggregates and charged variants.

The first part of the pH/buffer screen included the three buffer systems L-histidine/L-histidine-HCl (His/His-HCl), L-histidine-acetate (His/Acetate) and sodium acetate (Na/acetate), a pH range between 5.3 and 6.5, buffer strength ranges between 7 and 300 mM and an ionic strength range between 5 and 86. The setup of the active formulations is shown in table 3.

TABLE 3
Formulation codes: ph/buffer screen part I

| | | | Buffer | | | | |
|---|---|---|---|---|---|---|---|
| | | | Concen- | | | Fill | |
| Form. | Buffer | | tration | Ionic | Protein | volume | Dosage |
| Code | system | pH | (mM) | strength | (mg/ml) | (ml) | form |
| F1 | His/ | 5.3 | 7 | 5 | 120 | 2.7 | 6 mL |
| | His-HCl | | | | | | vial |
| F2 | | 5.9 | 75 | 45.5 | 120 | 2.7 | 6 mL |
| | | | | | | | vial |
| F3 | | 6.5 | 300 | 86 | 120 | 2.7 | 6 mL |
| | | | | | | | vial |
| F4 | His/ | 5.3 | 102 | 86 | 120 | 2.7 | 6 mL |
| | Acetate | | | | | | vial |
| F5 | | 5.9 | 10 | 5 | 120 | 2.7 | 6 mL |
| | | | | | | | vial |
| F6 | | 6.5 | 180 | 45.5 | 120 | 2.7 | 6 mL |
| | | | | | | | vial |
| F7 | Na/ | 5.3 | 58 | 45.5 | 120 | 2.7 | 6 mL |
| | Acetate | | | | | | vial |
| F8 | | 5.9 | 91 | 86 | 120 | 2.7 | 6 mL |
| | | | | | | | vial |
| F9 | | 6.2 | 6 | 5 | 120 | 2.7 | 6 mL |
| | | | | | | | vial |
| F10 | His/ | 5.3 | 100 | 86 | 120 | 2.7 | 6 mL |
| | His-HCl | | | | | | vial |
| F11 | | 5.9 | 10 | 5 | 120 | 2.7 | 6 mL |
| | | | | | | | vial |
| F12 | | 6.5 | 165 | 45.5 | 120 | 2.7 | 6 mL |
| | | | | | | | vial |
| F13 | His/ | 5.3 | 53 | 45.5 | 120 | 2.7 | 6 mL |
| | Acetate | | | | | | vial |
| F14 | | 5.9 | 150 | 86 | 120 | 2.7 | 6 mL |
| | | | | | | | vial |
| F15 | | 6.5 | 20 | 5 | 120 | 2.7 | 6 mL |
| | | | | | | | vial |
| F16 | Na/ | 5.3 | 7 | 5 | 120 | 2.7 | 6 mL |
| | Acetate | | | | | | vial |
| F17 | | 5.9 | 49 | 45.5 | 120 | 2.7 | 6 mL |
| | | | | | | | vial |
| F18 | | 6.2 | 89 | 86 | 120 | 2.7 | 6 mL |
| | | | | | | | vial |
| F19 | His/ | 5.9 | 75 | 45.5 | 120 | 2.7 | 6 mL |
| | His-HCl | | | | | | vial |
| F20 | | 5.9 | 75 | 45.5 | 120 | 2.7 | 6 mL |
| | | | | | | | vial |

### Material and Methods

A summary of materials used during the preparation of the formulations and their primary packaging is given Table 1 and Table 2.

The drug substance was buffer exchanged by dialysis using a Slide-A-Lyzer™ G2 (Thermo Scientific) with a 10 kD molecular weight cut-off against the buffer systems as listed in Table 3. Thereby, 42 mL of drug substance was filled into the dialysis device and buffer exchanged against three times 5 L of dialysis buffer.

Optionally, if the protein concentration was below 120 mg/mL after dialysis, the drug substance was concentrated by centrifugation using an Amicon® Ultra 15, Ultracel 10K (Millipore) device (20° C., 4000 rpm).

Afterwards, the dialyzed and optionally concentrated drug substance was diluted with the respective dialysis buffer to a target protein concentration of 120 mg/mL, resulting the final drug product solution.

Each drug product solution was filtered through a 0.22 μm Sterivex GV (Millipore) filter and is filled into clean and sterile 6 mL vials with a fill volume of 2.7 mL. The vials were stoppered and crimped.

### Analytical Methods

The analytical test methods turbidity and viscosity are described in example 1.

### Results

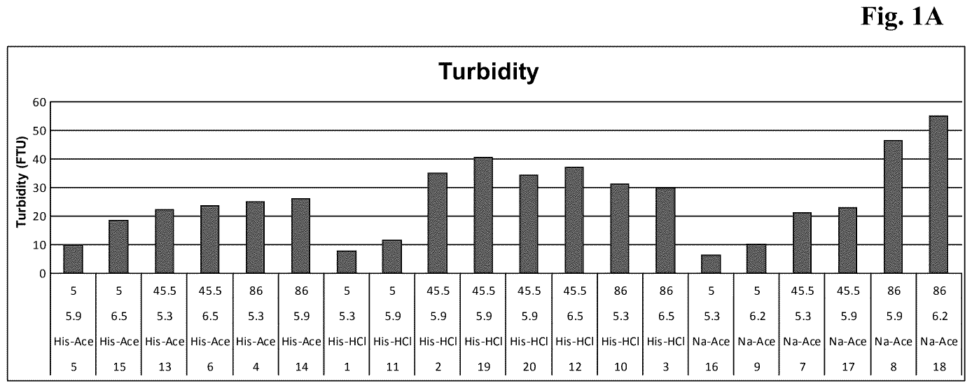

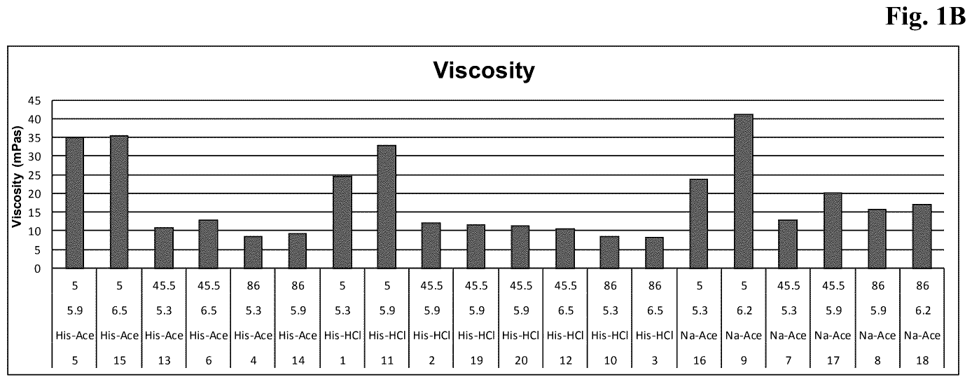

FIG. 1 compares the turbidity and the viscosity results of the formulations from the pH/buffer screen I.

In general, formulations with high ionic strength (e.g. 4, 14, 10, 3, 8 and 18) show a high turbidity (above 30 FTU) and a low viscosity (approximately 10 mPas). Instead, formulations with a low ionic strength have a high viscosity (approximately 25 to 40 mPas) and a low turbidity (below 10 FTU). Surprisingly, formulations with the buffer system histidine-acetate (13, 6, 4, 14) could be identified with low viscosities of approximately 10 mPas and a turbidity of less than 25 FTU.

### Conclusion

Formulations from the pH/buffer screen I were measured for viscosity and turbidity. Surprisingly, formulations of the histidine-acetate buffer system and with an ionic strength of 45.5 or higher showed low viscosity and reduced turbidity values.

### Example 3: PH/Buffer Screen II

### Set-Up

The scope of the pH/buffer screen was to select the optimal pH and buffer for the formulation of anti-VEGF/ANG2 antibody CrossMAb VEGFang2-0016 (faricimab) anti-VEGF/ANG2 antibody and to select a formulation with a low viscosity, a reduced turbidity and a good stability behavior resulting in a low formation of soluble aggregates and charged variants.

The second part of the pH/buffer screen was designed based on the outcome of the pH/buffer screen I and included the buffer system L-histidine-acetate (His/Acetate), a pH range between 5.5 and 6.0 with a buffer strength range between 14 and 59 mM. The ionic strength of the formulations was modified either by increasing the buffer strength or by addition of sodium chloride (NaCl) or Calcium chloride (Ca Cl₂) resulting in ionic strength range between 10 and 50. The setup of the active formulations is shown in table 4.

TABLE 4
Formulation codes: ph/buffer screen part II

| Form. | | Buffer | | | Salt | | | | |
|---|---|---|---|---|---|---|---|---|---|
| Code | | Concen- | | | concen- | | | Fill | |
| (= -sample | Buffer | tration | | | tration | Ionic | Protein | volume | Dosage |
| no) | system | (mM) | pH | Salt | (mM) | strength | (mg/ml) | (ml) | form |
| GRM0069- | His- | 14 | 5.5 | — | — | 10 | 120 | 2.7 | 6-mL |
| 01 = 01 | Acetate | | | | | | | | vial |
| GRM0069- | His- | 39 | 5.5 | — | — | 30 | 120 | 2.7 | 6-mL |
| 02 = 02 | Acetate | | | | | | | | vial |
| GRM0069- | His- | 14 | 5.5 | NaCl | 20 | 30 | 120 | 2.7 | 6-mL |
| 03 = 03 | Acetate | | | | | | | | vial |
| GRM0069- | His- | 14 | 5.5 | CaCl₂ | 6.7 | 30 | 120 | 2.7 | 6-mL |
| 04 = 04 | Acetate | | | | | | | | vial |
| GRM069- | His- | 14 | 5.5 | NaCl | 40 | 50 | 120 | 2.7 | 6-mL |
| 05 = 05 | Acetate | | | | | | | | vial |
| GRM0069- | His- | 14 | 5.5 | CaCl₂ | 13.3 | 50 | 120 | 2.7 | 6-mL |
| 06 = 06 | Acetate | | | | | | | | vial |
| GRM0069- | His- | 20 | 6.0 | — | — | 10 | 120 | 2.7 | 6-mL |
| 07 = 07 | Acetate | | | | | | | | vial |
| GRM0069- | His- | 59 | 6.0 | — | — | 30 | 120 | 2.7 | 6-mL |
| 08 = 08 | Acetate | | | | | | | | vial |
| GRM0069- | His- | 20 | 6.0 | NaCl | 20 | 30 | 120 | 2.7 | 6-mL |
| 09 = 09 | Acetate | | | | | | | | vial |
| GRM0069- | His- | 20 | 6.0 | CaCl₂ | 6.7 | 30 | 120 | 2.7 | 6-mL |
| 10 = 10 | Acetate | | | | | | | | vial |
| GRM0069- | His- | 20 | 6.0 | NaCl | 40 | 50 | 120 | 2.7 | 6-mL |
| 11 = 11 | Acetate | | | | | | | | vial |
| GRM0069- | His- | 20 | 6.0 | CaCl₂ | 13.3 | 50 | 120 | 2.7 | 6-mL |
| 12 = 12 | Acetate | | | | | | | | vial |

### Material and Methods

A summary of materials used during the preparation of the formulations and their primary packaging is given Table 1 and Table 2.

The drug substance was buffer exchanged by ultrafiltration-diafiltration using a Labscale TTF (Millipore) with a 30 kD molecular weight cut-off semi-permeable membrane against the buffer systems as listed in Table 4. Thereby, 120 mL of drug substance was filled into the Labscale system and buffer exchanged against 1050 mL of diafiltration buffer.

After buffer-exchange the drug substance was concentrated in the Labscale system to a protein concentration of approximately 150 mg/mL.

Afterwards, the concentrated drug substance was diluted with stock solutions of respective buffer and salt solutions to a target protein concentration of 120 mg/mL, resulting the final drug product solution according to table 4.

Each drug product solution was filtered through a 0.22 μm Sterivex GV (Millipore) filter and is filled into clean and sterile 6 mL vials with a fill volume of 2.7 mL. The vials were stoppered and crimped.

### Analytical Methods

The analytical test methods turbidity, viscosity and SE-HPLC are described in example I.

### Stability Program

The formulations were kept on stability at 2-8° C. and 25 for 8 weeks. Samples were drawn and analyzed at start of the stability study and after 8 weeks' storage.

### Results

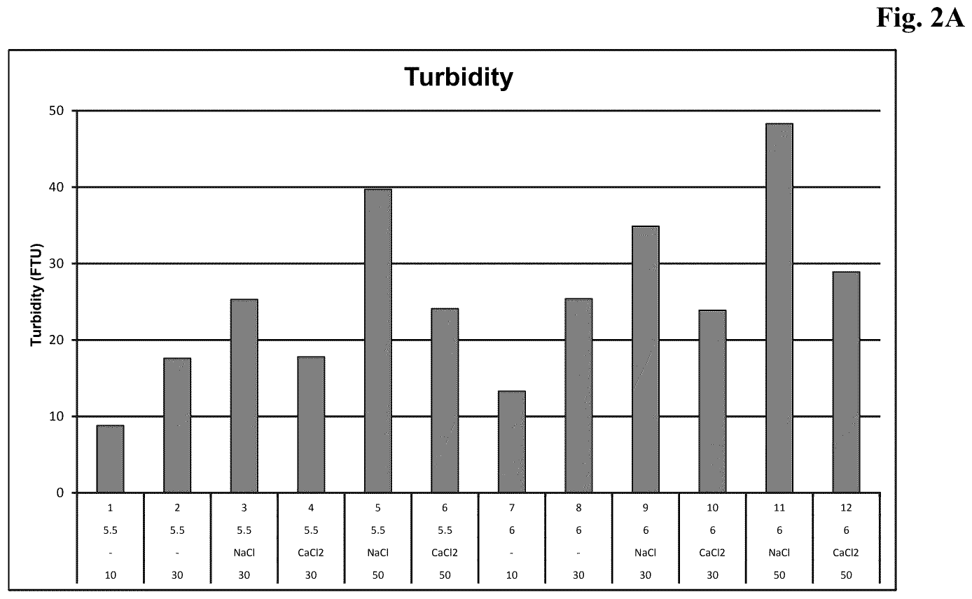

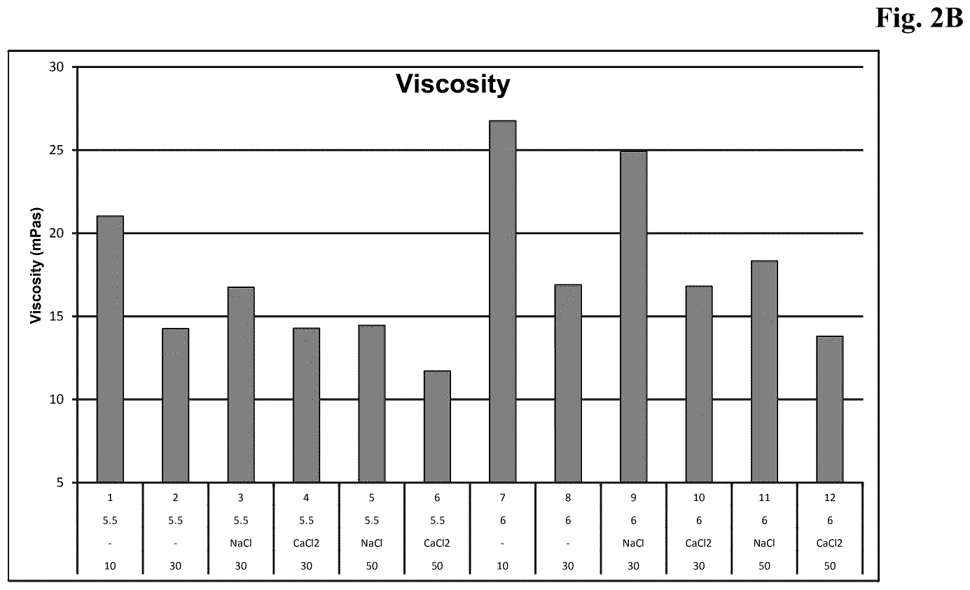

FIG. 2 compares the turbidity and viscosity results of the second part of the pH/buffer screen. In general, turbidity is increasing with increasing ionic strength and with a pH of 6.0 in comparison to a pH of 5.5. In addition, the presence of sodium chloride leads also to a higher turbidity. A pH of 5.5 also reduces the viscosity in comparison to a pH of 6. Furthermore, an ionic strength of at least 30 helps to reduce the viscosity to a level of approximately 15 mPas. When comparing the effect of the different salts on viscosity, calcium chloride or a higher buffer strength are more efficient to reduce viscosity than sodium chloride.

Considering both goals, reducing turbidity and viscosity, a formulation with an ionic strength of at least 30 and a pH of 5.5 shows a reduced turbidity level of approximately 20 FTU and a viscosity around 15 mPas.

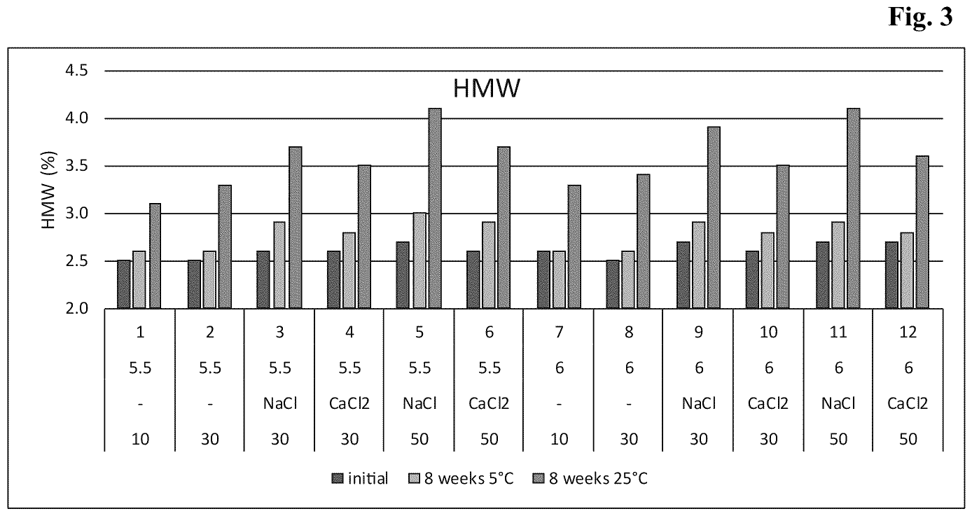

FIG. 3 compares the aggregate levels (HMWS) of the pH/buffer screen II formulations at start (=0) with the levels after 8 weeks at 5° C. and 25° C. In general, the HMW levels increase with increasing ionic strength. Comparing the effect of the different salts, a high buffer strength or the presence of sodium chloride leads to a lower increase of aggregates than the presence of sodium chloride. A lower pH of 5.5 has a small impact on reducing the aggregate formation.

### Conclusion

A formulation with a pH of 5.5 and an ionic strength of 30 provides an optimum with reduced turbidity, low viscosity and reduced aggregate formation. The ionic strength can be adjusted with a higher buffer strength, or the addition of the salts sodium chloride and calcium chloride. In general, a higher buffer strength or the presence of calcium chloride show a preferred turbidity and viscosity behavior.

### Example 4: Surfactant Screen

### Set-Up

The scope of the surfactant screen is to select the optimal surfactant type and the surfactant concentration for the formulation of anti-VEGF/ANG2 antibody CrossMAb VEGFang2-0016 (faricimab).

A formulation matrix of 120 mg/mL VEGF/Ang-2 antibody, 20 mM L-histidine-acetate buffer at pH 5.5, 25 mM sodium chloride and 180 mM sucrose based on the outcome of the pH/buffer screens I and II and to enable an isotonic formulation with a target osmolality of 300±50 mOsm/kg.

The surfactant screen tested the stabilizing effect of the surfactants polysorbate 20 and poloxamer 188 at different surfactant concentrations between 0.01 and 0.07% on the Vegf-Ang2 antibody. In addition, a surfactant-free formulation was tested.

Table 5 summarizes the test formulations of the surfactant screen.

TABLE 5
Formulation codes for the surfactant screen

| Form. | | | | | | | |
|---|---|---|---|---|---|---|---|
| Code | | | Target | | Surfactant | Fill | |
| (= -sample | Formulation | Ionic | Osmolality | | concentration | volume | Dosage |
| no) | matrix | strength | (mOsm/kg) | Surfactant | (%) | (ml) | form |
| GRM0071- | 120 mg/mL | 40 | 311 | — | 0 | 2.7 | 6-mL |
| 01 | Vegf-Ang2 | | | | | | vial |
| GRM0071- | antibody | | | Polysorbate | 0.01 | 2.7 | 6-mL |
| 02 | 20 mM | | | 20 | | | vial |
| GRM0071- | His-Acetate | | | Polysorbate | 0.03 | 2.7 | 6-mL |
| 03 | pH 5.5, | | | 20 | | | vial |
| GRM0071- | 25 mM | | | Polysorbate | 0.05 | 2.7 | 6-mL |
| 04 | NaCl | | | 20 | | | vial |
| GRM0071- | 180 mM | | | Polysorbate | 0.07 | 2.7 | 6-mL |
| 05 | sucrose | | | 20 | | | vial |
| GRM0071- | | | | Poloxamer | 0.01 | 2.7 | 6-mL |
| 06 | | | | | | | vial |
| GRM0071- | | | | Poloxamer | 0.03 | 2.7 | 6-mL |
| 07 | | | | | | | vial |
| GRM0071- | | | | Poloxamer | 0.05 | 2.7 | 6-mL |
| 08 | | | | | | | vial |
| GRM0071- | | | | Poloxamer | 0.07 | 2.7 | 6-mL |
| 09 | | | | | | | vial |

### Material and Methods

A summary of materials used during the preparation of the formulations and their primary packaging is given Table 1 and Table 2.

The drug substance was buffer exchanged by ultrafiltration-diafiltration using a Labscale TTF (Millipore) with a 30 kD molecular weight cut-off semi-permeable membrane against the 20 mM histidine-acetate pH 5.3 diafiltration buffer. Thereby, 250 mL of drug substance was filled into the Labscale system and buffer exchanged against 1700 mL of diafiltration buffer.

After buffer-exchange the drug substance was concentrated in the Labscale system to a protein concentration of approximately 170 mg/mL and a pH of approximately 5.5.

Afterwards, the concentrated drug substance was diluted with stock solutions of respective buffer and salt solutions to a target protein concentration of 120 mg/mL, resulting the final drug product solution according to table 5.

Each drug product solution was filtered through a 0.22 μm Sterivex GV (Millipore) filter and is filled into clean and sterile 6 mL vials with a fill volume of 2.7 mL. The vials were stoppered and crimped.

### Analytical Methods

The analytical test methods protein concentration, pH, osmolality, turbidity, viscosity, visible particles and SE-HPLC are described in example I.

### Stability Program

During the surfactant screen mechanical stress test conditions of 1-week horizontal shaking at 2-8° C. (200 rpm), 1 week horizontal shaking at 25° C. (200 rpm) and 5 cycles Freeze/Thaw (−40° C./5° C.) were applied.

### Results

Table 6 summarizes the initial results of the surfactant screen samples. All formulations had a protein concentration of approximately 120 mg/mL and a pH of 5.5±0.1. The measured osmolality was between 335 and 350 mOsm/kg and thereby slightly higher than the target of 311 mOsm/kg. The selected formulation matrix of 120 mg/mL Vegf-Ang2 with a 20 mM histidine-acetate buffer at pH 5.5 plus 25 mM sodium chloride and 180 mM sucrose resulted in low viscosities of approximately 15 mPas and reduced turbidity of 20 FTU.

The surfactant screen formulations were exposed to shaking stress at 5° C. and 25° C. and freeze-thaw stress (five freeze-thaw cycles) and were analyzed for visible particles and soluble aggregates (HMWS).

Table 7 summarizes the visible particles results at initial and after physical stresses. All formulation samples were free of particles at initial. After exposing to the different physical stresses, the formulation GRM0071-01 without surfactant showed always many particles. The addition of at least 0.01% polysorbate 20 prevented the formation of visible particles during exposure to the three physical stress methods.

Surprisingly, the addition of poloxamer could not prevent the formation of visible particles after exposure to 1 week shaking at 5° C., whereas it was able to protect the protein against shaking at 25° C. and freeze-thaw stress.

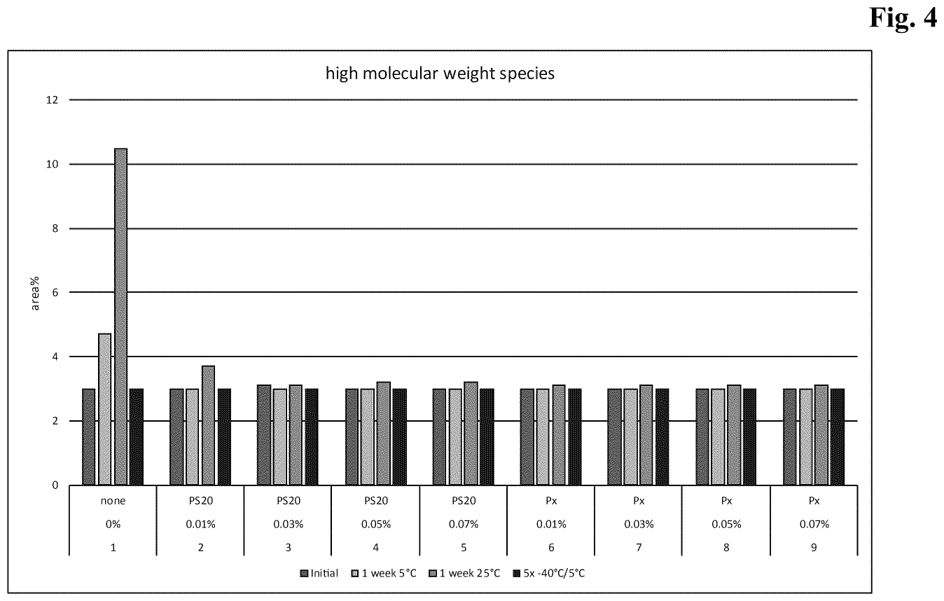

FIG. 4 shows the soluble aggregate levels (HMWS) of the initial samples and the stressed samples. The formulation GRM0071-01 (1) without surfactant was sensitive to shaking stress and showed increased aggregate levels up to 10% after 1 week shaking at 5° C. and 25° C. The presence of 0.01% polysorbate 20 was not sufficient to fully prevent the increase of soluble aggregates after 1 week shaking at 25° C. as the aggregate level increased from 3 to 3.8%. Surprisingly, a polysorbate 20 level of equal or higher than 0.03% was required to prevent an increase of soluble aggregates after 1 week shaking at 25° C.

TABLE 6
Summary on protein concentration, pH, osmolality, turbidity and
viscosity results of the surfactant screen samples after manufacturing

| Form. Code | Protein | | | | |
|---|---|---|---|---|---|
| (= -sample | concentration | | Osmolality | Turbidity | Viscosity |
| no) | (mg/mL) | pH | (mOsm/kg) | (FTU) | (mPas) |
| GRM0071-01 | 121 | 5.6 | 341 | 22.9 | 15 |
| GRM0071-02 | 118 | 5.6 | 344 | 20.4 | 14 |
| GRM0071-03 | 121 | 5.6 | 337 | 20 | 14 |
| GRM0071-04 | 124 | 5.5 | 341 | 20.1 | 14 |
| GRM0071-05 | 121 | 5.5 | 341 | 20.3 | 14 |
| GRM0071-06 | 121 | 5.5 | 347 | 20.5 | 14 |
| GRM0071-07 | 120 | 5.6 | 340 | 20.1 | 15 |
| GRM0071-08 | 121 | 5.5 | 350 | 21 | 15 |
| GRM0071-09 | 122 | 5.5 | 346 | 20.8 | 15 |

TABLE 7
Summary on visible particle results of initial and physically stressed
surfactant screen samples

| | | | after | after | |
|---|---|---|---|---|---|
| Form. Code | | | 1 week | 1 week | after 5 |
| (=-sample | | | shaking | shaking | freeze/thaw |
| no) | Surfactant | Initial | at 5° C. | at 25° C. | cycles |
| GRM0071-01 | none | free of | with many | with many | with many |
| | | particles | particles | particles | particles |
| GRM0071-02 | 0.01% | free of | free of | free of | free of |
| | Polysorbate | particles | particles | particles | particles |
| | 20 | | | | |
| GRM0071-03 | 0.03% | free of | free of | free of | free of |
| | Polysorbate | particles | particles | particles | particles |
| | 20 | | | | |
| GRM0071-04 | 0.05% | free of | free of | free of | free of |
| | Polysorbate | particles | particles | particles | particles |
| | 20 | | | | |
| GRM0071-05 | 0.07% | free of | free of | free of | free of |
| | Polysorbate | particles | particles | particles | particles |
| | 20 | | | | |
| GRM0071-06 | 0.01% | free of | with many | free of | free of |
| | Poloxamer | particles | particles | particles | particles |
| GRM0071-07 | 0.03% | free of | with many | free of | free of |
| | Poloxamer | particles | particles | particles | particles |
| GRM0071-08 | 0.05% | free of | with many | free of | free of |
| | Poloxamer | particles | particles | particles | particles |
| GRM0071-09 | 0.07% | free of | with many | free of | free of |
| | Poloxamer | particles | particles | particles | particles |

### Conclusion

The addition of at least 0.03% polysorbate 20 is required to fully stabilize anti-VEGF/ANG2 antibody at a concentration of 120 mg/mL against shaking and freeze-thaw stress.

The surfactant poloxamer is not able to protect the bispecific anti-VEGF/ANG2 antibody at a concentration of 120 mg/mL against shaking stress at 5° C.

The formulation matrix with 20 mM histidine-acetate buffer at pH 5.5, 25 mM sodium chloride and 180 mM sucrose provides acceptable turbidity (approximately 20 FTU) and viscosity results (approximately 15 mPas) for a 120 mg/mL anti-VEGF/ANG2 antibody formulation.

### Example 5: Excipient Screen I

### Set-Up

The scope of the excipient screen is to select the final composition for the commercial formulation of the anti-VEGF/ANG2 antibody CrossMAb VEGFang2-0016 (faricimab).

Based on the outcome of the previous pH/buffer screens I and II and the surfactant screen, a formulation matrix was selected which consisted of 120 mg/mL Vegf-Ang2 antibody, a 20 mM histidine acetate buffer system, 160 mM sucrose and 0.04% polysorbate 20. In the formulation matrix the effect of pH (5.5 versus 5.8), salt (25 mM sodium chloride versus 8 mM calcium chloride) and methionine (0 versus 7 mM) was tested. The ionic strength was adjusted to 40 based on the contribution from the buffer and salt concentration.

Table 8 summarizes the formulations of the excipient screen I.

TABLE 8
Formulation codes for the excipient screen part I

| Form. | | | | | | | | |
|---|---|---|---|---|---|---|---|---|
| Code | Protein | | | | | | | |
| (= -sample | concen- | pH/ | | Methionine | | Polysorbate | Fill | Dosage |
| no) | tration | buffer | Salt | (mM) | Sucrose | 20 | volume | Form |
| GRM0073- | 120 | 20 mM | 25 mM | 0 | 160 mM | 0.04% | 2.7 mL | 6-mL |
| 01 | mg/mL | Histidine- | NaCl | | | | | vial |
| GRM0073- | | acetate | | 7 | | | | |
| 02 | | pH 5.5 | | | | | | |
| GRM0073- | | | 8 mM | 0 | | | | |
| 03 | | | CaCl₂ | | | | | |
| GRM0073- | | | | 7 | | | | |
| 04 | | | | | | | | |
| GRM0073- | | 20 mM | 25 mM | 0 | | | | |
| 05 | | Histidine- | NaCl | | | | | |
| GRM0073- | | acetate | | 7 | | | | |
| 06Tab | | pH 5.8 | | | | | | |
| GRM0073- | | | 8 mM | 0 | | | | |
| 07 | | | CaCl₂ | | | | | |
| GRM0073- | | | | 7 | | | | |
| 08 | | | | | | | | |

### Material and Methods

A summary of materials used during the preparation of the formulations and their primary packaging is given Table 1 and Table 2.

The drug substance was buffer exchanged by ultrafiltration-diafiltration using a Labscale TTF (Millipore) with a 30 kD molecular weight cut-off semi-permeable membrane against either 20 mM histidine-acetate pH 5.3 diafiltration buffer or 20 mM histidine-acetate pH 5.6. Thereby, 410 mL of drug substance was filled into the Labscale system and buffer exchanged against 3000 mL of diafiltration buffer.

After buffer-exchange the drug substance was concentrated in the Labscale system to a protein concentration of approximately 165 mg/mL and a pH of either approximately 5.5 or pH 5.8.

Afterwards, the concentrated drug substance was diluted with stock solutions of respective buffer and salt solutions to a target protein concentration of 120 mg/mL, resulting the final drug product solution according to table 8.

Each drug product solution was filtered through a 0.22 μm Sterivex GV (Millipore) filter and was filled into clean and sterile 6 mL vials with a fill volume of 2.7 mL. The vials were stoppered and crimped.

### Analytical Methods

The analytical test methods protein concentration, pH, osmolality, turbidity, viscosity, visible particles, SE-HPLC and IE-HPLC are described in example I.

### Stability Program

The formulations were kept on stability at 2-8° C. for up to 20 weeks and at 25° C. for up to 13-weeks. In addition, samples were exposed to 1-week horizontal shaking at 2-8° C. (200 rpm), 1-week horizontal shaking at 25° C. (200 rpm) and 5 cycles Freeze/Thaw (−40° C./5° C.).

### Results

Table 9 summarizes the initial results of the excipient screen I. All formulations had a protein concentration between 125 and 130 mg/mL. The formulations GRM0073-01 to -04 with a target pH of 5.5 had measured pH values of approximately 5.6, whereas the formulations GRM0073-05 to -08 with a target pH of 5.8 had a measured pH values of approximately 5.9. The osmolality of the formulations containing 25 mM sodium chloride (GRM0073-01, -02, -05 and -06) had higher osmolality results (between 313 and 322 mOsm/kg) in comparison to the formulations with 8 mM calcium chloride (GRM0073-03, -04, -07 and -08), which were between 273 and 288 mOsm/kg).

Table 10 summarizes the visible particle results at initial, after physical stress and after 13-weeks storage at 5° C. and 25° C. After manufacturing and after exposure to physical stress (1 week shaking at 5° C. or 25° C., or five freeze-thaw cycles), all formulations were free of particles. Surprisingly, all formulations which contained 8 mM calcium chloride showed visible particles after 13 weeks' storage at 5° C. and 25° C., whereas all formulations which container 25 mM sodium chloride were free of particles.

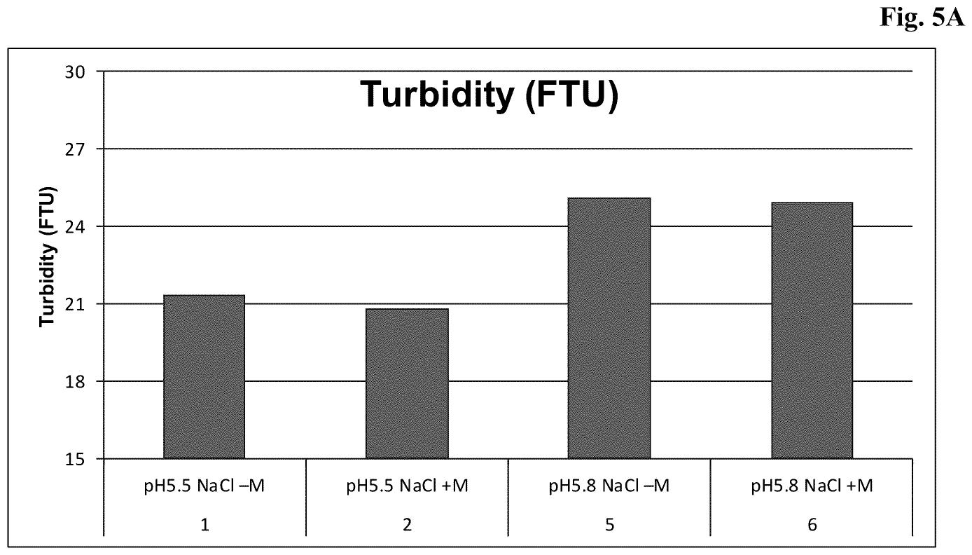

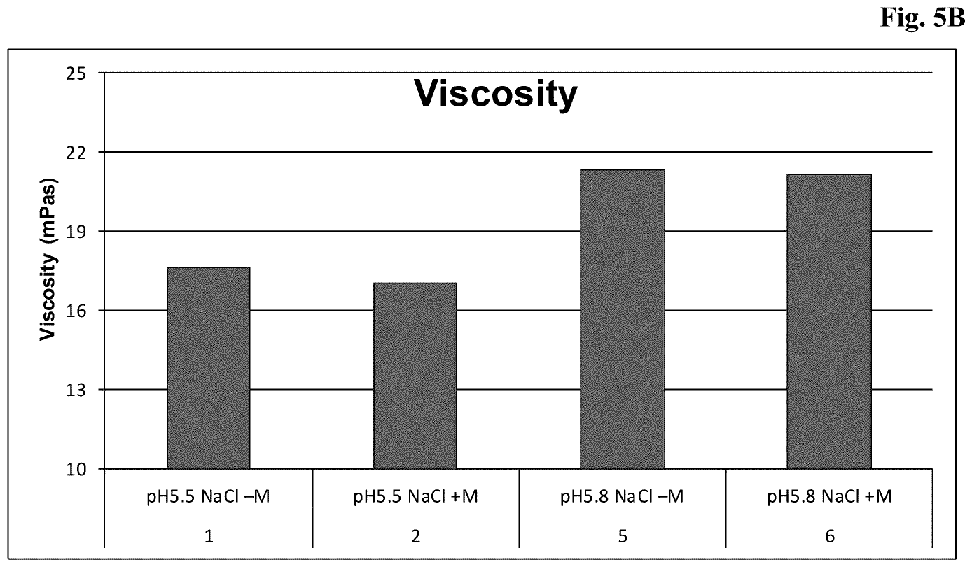

FIG. 5 compares turbidity and viscosity results of the formulations containing 25 mM sodium chloride. Thereby, the formulations with a pH of 5.5 showed lower turbidity (21 FTU versus 25 FTU) and lower viscosity (17 mPas versus 21 mPas).

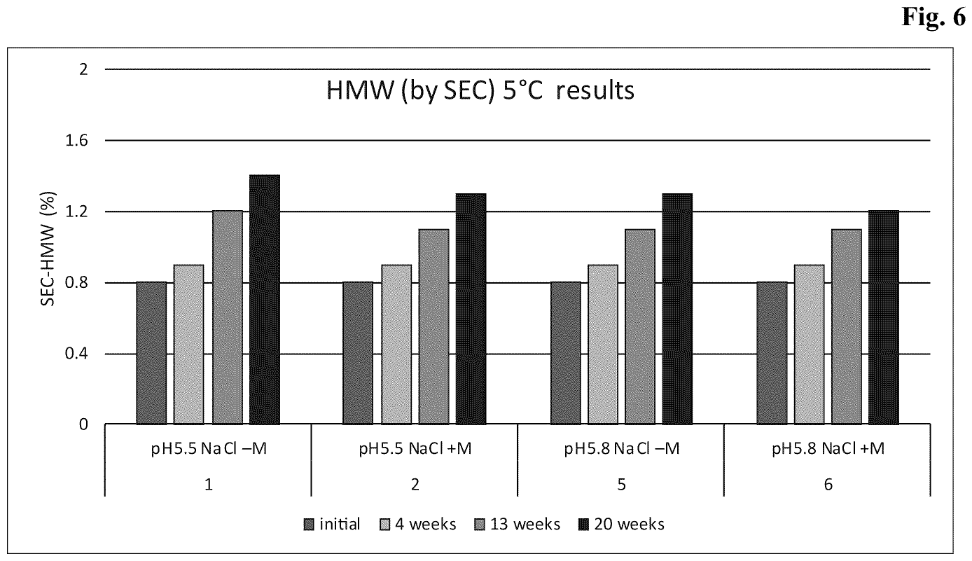

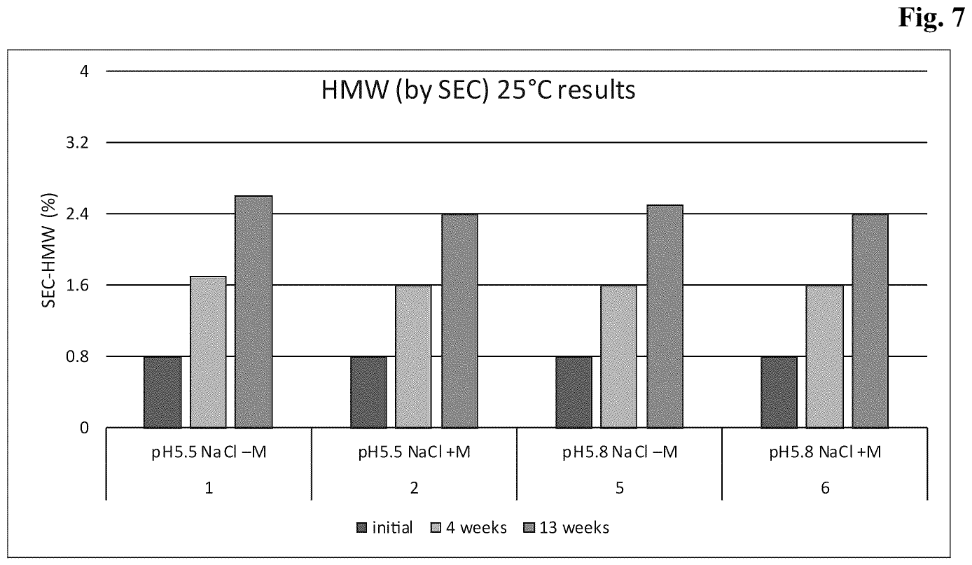

FIGS. 6 and 7 show the increase of soluble aggregates (HMWS) during 20 weeks' storage at 5° C., respectively 13 weeks storage at 25° C. The formulations with a pH of 5.8 showed a slightly lower increase in soluble aggregates in comparison to the formulations at pH 5.5. Surprisingly, the addition of methionine could reduce the formation of soluble aggregates and could compensate the influence of a lower pH on aggregation.

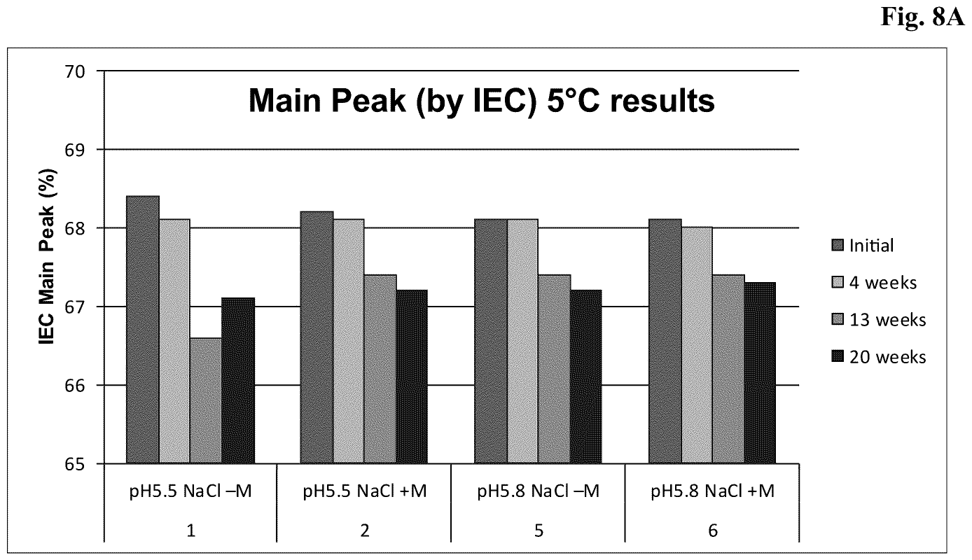

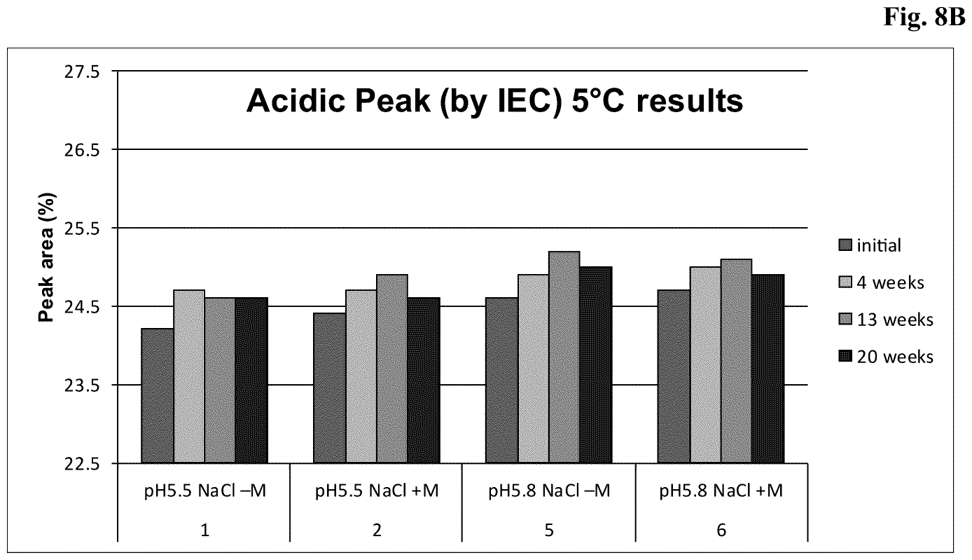

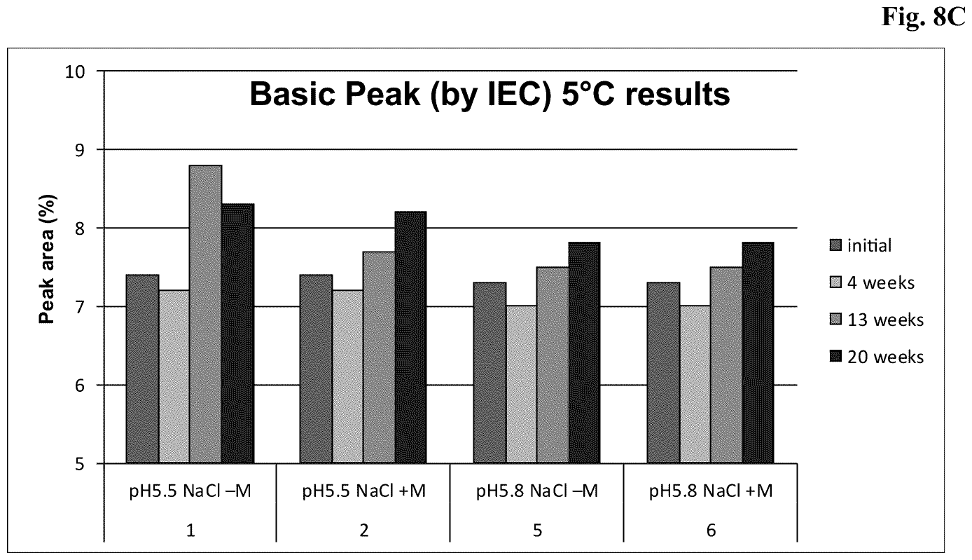

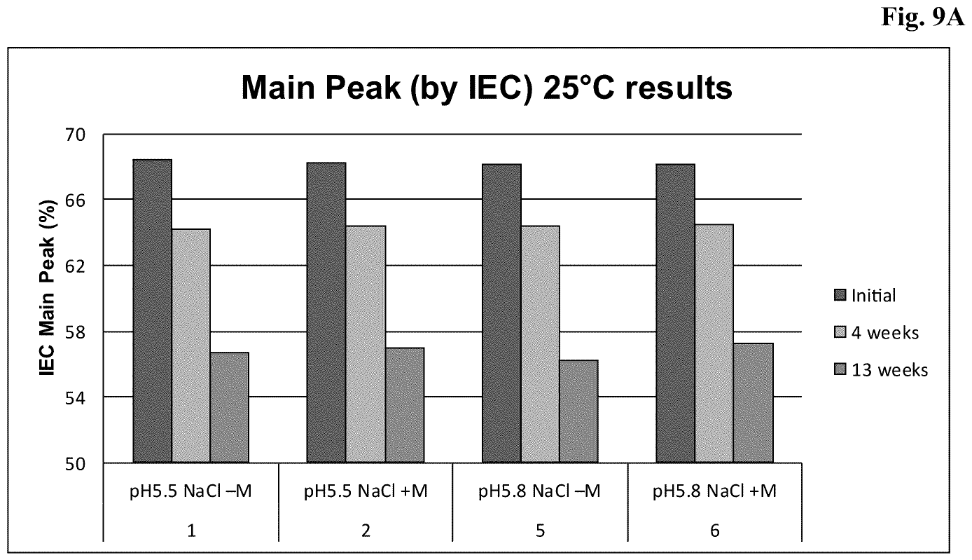

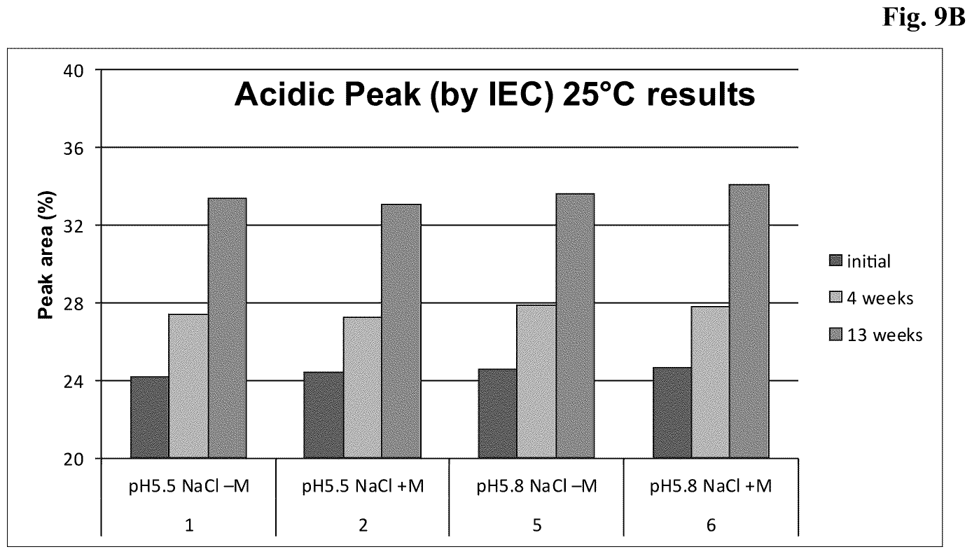

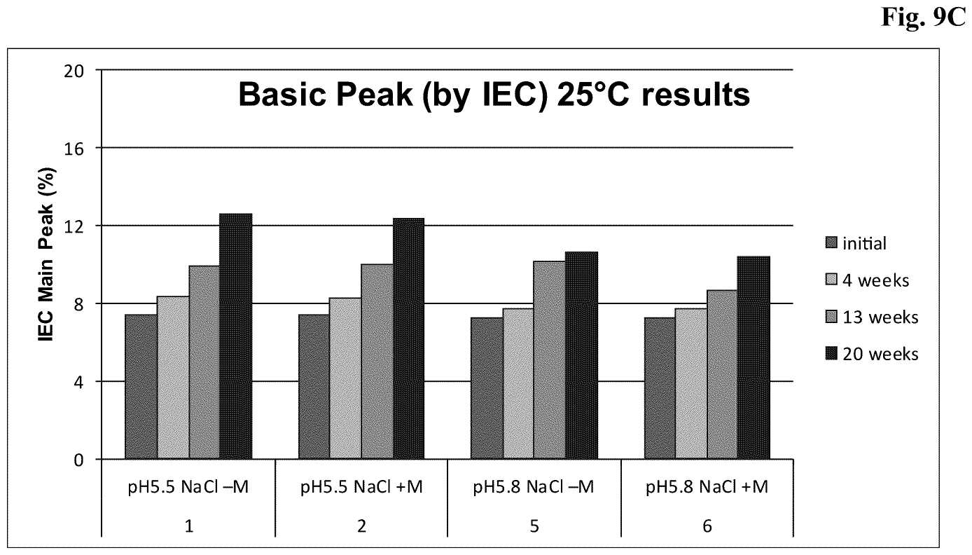

FIGS. 8 and 9 compare the change of charged variants measured by IEC after 20 weeks' storage at 5° C. and 13 weeks storage at 25° C., respectively. All formulations showed a slight drop of approximately 1% of the main peak area after 13 weeks storage at 5° C. This is accompanied by an approximate 0.2% increase in acidic variants and an approximate 1% increase in basic peak area. There is no clear differentiation caused by the different pH or the presence of methionine. Although the drop in main peak is much stronger (approximately 18%), after 13 weeks storage at 25° C., there is no clear differentiation based on pH and methionine. The decrease in main peak during storage at 25° C. is mainly caused by an increase in acidic variants.

TABLE 9
Summary on protein concentration, pH and osmolality results
of the excipient screen I samples after manufacturing

| Form. Code | Protein concentration | | Osmolality |
|---|---|---|---|
| (= -sample no) | (mg/mL) | pH | (mOsm/kg) |
| GRM0073-01 | 126 | 5.6 | 315 |
| GRM0073-02 | 125 | 5.6 | 322 |
| GRM0073-03 | 126 | 5.6 | 275 |
| GRM0073-04 | 127 | 5.6 | 288 |
| GRM0073-05 | 126 | 5.9 | 313 |
| GRM0073-06 | 128 | 5.9 | 314 |
| GRM0073-07 | 129 | 5.9 | 273 |
| GRM0073-08 | 128 | 5.9 | 281 |

TABLE 10
Summary on visible particle results of initial and stressed excipient
screen I samples

| | | | after | after | after 5 | | |
|---|---|---|---|---|---|---|---|
| Form. Code | | | 1 week | 1 week | freeze/ | after | after |
| (= -sample | | | shaking | shaking | thaw | 13 weeks | 13 weeks |
| no) | Salt | Initial | at 5° C. | at 25° C. | cycles | at 5° C. | at 25° C. |
| GRM0073- | 25 mM | free of | free of | free of | free of | free of | free of |
| 01 | NaCl | particles | particles | particles | particles | particles | particles |
| GRM0073- | | free of | free of | free of | free of | free of | free of |
| 02 | | particles | particles | particles | particles | particles | particles |
| GRM0073- | 8 mM | free of | free of | free of | free of | with many | with many |
| 03 | CaCl₂ | particles | particles | particles | particles | particles | particles |
| GRM0073- | | free of | free of | free of | free of | with many | with many |
| 04 | | particles | particles | particles | particles | particles | particles |
| GRM0073- | 25 mM | free of | free of | free of | free of | free of | free of |
| 05 | NaCl | particles | particles | particles | particles | particles | particles |
| GRM0073- | | free of | free of | free of | free of | free of | free of |
| 06 | | particles | particles | particles | particles | particles | particles |
| GRM0073- | 8 mM | free of | free of | free of | free of | with many | with many |
| 07 | CaCl₂ | particles | particles | particles | particles | particles | particles |
| GRM0073- | | free of | free of | free of | free of | with many | with many |
| 08 | | particles | particles | particles | particles | particles | particles |

### Conclusion

Although the presence of calcium chloride leads to lower viscosity and turbidity levels in comparison to formulations with sodium chloride (refer to examples 2 and 3), it surprisingly also caused the formation of visible particles. The formation of visible particles is not acceptable for an intravitreal injection according to the requirements of USP-NF <790>, which is essentially free of visible particles. Therefore, the addition of sodium chloride as an ionic strength modifier for reduction of viscosity is preferred over the use of calcium chloride.

Furthermore, formulations at a pH of 5.5 show lower turbidity and lower viscosity in comparison to formulations at pH 5.8. However, formulations at a pH of 5.8 showed slightly less formation of soluble aggregates than formulations at pH 5.5, but this effect can be compensated by the addition of 7 mM methionine. Therefore, the addition of methionine allows to reduce soluble aggregate formation at pH 5.5 while still realizing lower viscosity and turbidity levels.

The difference in pH (pH 5.5 versus 5.8) or the presence or absence of methionine have no impact on the formation of charged variants.

In summary, a formulation with 25 mM sodium chloride instead of 8 mM calcium chloride and a 20 mM histidine-acetate buffer at pH 5.5 with 7 mM methionine and 160 mM sucrose and 0.04% polysorbate 20 allows a particle-free formulation with low turbidity and viscosity and an improved stability behavior.

### Example 6: Excipient Screen II

### Set-Up

In the second part of the excipient screen, the stability behavior is further characterized in a pre-filled syringe and at a protein concentration of 30 mg/mL.

The excipient screen I resulted in an optimized formulation consisting of 120 mg/mL anti-VEGF/ANG2 antibody CrossMAb VEGFang2-0016 (faricimab), 20 mM histidine-acetate at pH 5.5, 25 mM sodium chloride, 160 mM sucrose, 7 mM methionine and 0.04% polysorbate 20, which was filled in glass vial (corresponds to formulation GRM0073-02).

This formulation was also filled in pre-filled syringe (GRM0076-02). In addition, the stability behavior of this formulation matrix was tested at a protein concentration of 30 mg/mL, filled either in a pre-filled syringe (GRM0077-02) or in a glass vial (GRM0077-06).

For comparison, the stability of the reference formulation filled in a glass vial was tested at 30 mg/mL (GRM0077-09) and 120 mg/ml (GRM0076-05).

Table 11 summarizes the formulations of the excipient screen II.

TABLE 11
Formulation codes for the excipient screen part II

| | Protein | | | | | Poly- | | |
|---|---|---|---|---|---|---|---|---|
| Form. Code | concen- | pH/ | | | | sorbate | Fill | Dosage |
| (= sample no) | tration | buffer | Salt | Methionine | Sucrose | 20 | volume | Form |
| GRM0076- | 120 mg/mL | 20 mM | 25 mM | 7 mM | 160 mM | 0.04% | 1 mL | 1 mL |
| 02 | | Histidine- | NaCl | | | | | PFS |
| GRM0077- | 30 mg/mL | acetate | | | | | | |
| 02 | | pH 5.5 | | | | | | |
| GRM0077- | | | | | | | 2.7 | 6 mL |
| 06 | | | | | | | | Vial |
| GRM0077- | 30 mg/mL | 20 mM | 100 mM | 0 mM | 60 mM | | | |
| 09 | | Histidine- | NaCl | | | | | |
| GRM0076- | 120 mg/mL | HCl | | | | | | |
| 05 | | pH 6.0 | | | | | | |

### Material and Methods

A summary of materials used during the preparation of the formulations and their primary packaging is given Table 1 and Table 2.

The drug substance was buffer exchanged by ultrafiltration-diafiltration using a Labscale TTF (Millipore) with a 30 kD molecular weight cut-off semi-permeable membrane.

For the preparation of anti-VEGF/ANG2 antibody CrossMAb VEGFang2-0016 (faricimab) in 20 mM histidine-acetate buffer pH 5.5, approximately 340 mL of drug substance was filled into the Labscale system and buffer exchanged against 2400 mL of 20 mM histidine-acetate pH 5.2 diafiltration buffer.

Anti-VEGF/ANG2 antibody CrossMAb VEGFang2-0016 (faricimab) in 20 mM histidine-HCl buffer pH 6.0 was prepared using approximately 200 mL of drug substance. This was filled into the Labscale system and buffer exchanged against 1400 mL of 20 mM histidine-HCl pH 5.85 diafiltration buffer.

After buffer-exchange the respective drug substance was concentrated in the Labscale system to a protein concentration of approximately 165 mg/mL and a pH of either approximately 5.5 or pH 6.0.

Afterwards, the concentrated drug substance was diluted with stock solutions of respective buffer and salt solutions to a target protein concentration of 30 or 120 mg/mL, resulting the final drug product solution according to table 8.

Each drug product solution was filtered through a 0.22 μm Sterivex GV (Millipore) filter and was filled either into clean and sterile 6 mL vials with a fill volume of 2.7 mL or in clean sterilized 1 mL pre-filled syringes with a fill volume of 1 mL. The vials were stoppered and crimped, whereas the syringes were closed with a plunger stopper.

### Analytical Methods

The analytical test methods protein concentration, pH, osmolality, turbidity, viscosity, visible particles and SE-HPLC are described in example I.

### Stability Program

The formulations were kept on stability at 2-8° C. and 24° C. for up to 13 weeks. In addition, samples were exposed to 1-week horizontal shaking at 2-8° C. (200 rpm), 1-week horizontal shaking at 25° C. (200 rpm) and 5 cycles Freeze/Thaw (−40° C./5° C.).

### Results

Table 12 summarizes the initial results of the excipient screen II. GRM0076-02 (optimized formulation in PFS) and GRM0076-05 (reference formulation) matched both the targeted protein concentration of 120 mg/mL with measured values of 119 or 123 mg/mL, respectively. GRM0077-02 (optimized formulation in PFS), GRM0077-06 (optimized formulation in vial) and GRM0076-05 (reference formulation) were prepared at a target protein concentration of 30 mg/mL. The actual protein concentration were in the range between 30 and 31 mg/mL.

The pH pf all formulations was close to the targeted pH and with maximum deviation of just 0.1 pH units.

The osmolality of the formulations at 120 mg/mL were slightly higher and between 310 and 320 mOsm/kg, whereas the 30 mg/mL formulations were between 278 and 394 mOsm/kg.

Table 13 summarizes the visible particle results at initial, after physical stress and after 13-weeks storage at 5° C. and 25° C. After manufacturing and after exposure to physical stress (1 week shaking at 5° C. or 25° C., or five freeze-thaw cycles), all formulations were free of particles. Surprisingly, the reference formulations showed many particles after 13 weeks of storage at 5 and 25° C. All other formulations were free of particles.

Fig. compares turbidity and viscosity results of the of the optimized and the reference formulation at a protein concentration of 120 mg/mL. The optimized formulation showed a clearly lower turbidity of approximately 23 FTU, whereas the clinical service formulation had a turbidity of more than 45 FTU. Interestingly, the viscosity of both formulations were both below 14 mPas.

Fig. shows the turbidity and viscosity of the 30 mg/ml formulations. Here, the difference in turbidity is smaller between the formulations GRM0072-02 and GRM0077-09, but still the optimized formulations showed a lower turbidity. The viscosities for both 30 mg/mL formulations is very low compared to the 120 mg/mL formulations and is below 2 mPas.

Fig and Fig. show the increase of HMW species after 13 weeks storage at 5 and 25° C. The optimized formulation showed both at 120 mg/mL and 30 mg/mL less increase of HMW species than the reference formulation. The stabilizing effect is most pronounced after 13 weeks' storage at 25° C., where HMW increased only to 2.4% in the optimized formulation GRM0076-02, whereas the formulation GRM0076-05 showed an increase up to 2.9%. The same trend is also observed for the 30 mg/mL formulations, with a lower increase to 1.0% seen with the optimized formulation and an increase to 1.3% for the reference form.

Fig. compares the change of charged variants measured by IEC after 13 weeks storage at 5° C. and 25° C. All formulations showed a slight drop of approximately 1% of the main peak area after 13 weeks' storage at 5° C. This is accompanied by a corresponding increase in basic peak area, whereas the acidic peak area remains constant. Although the drop in main peak is much stronger (approximately 10%), after 13 weeks' storage at 25° C., there is no clear differentiation between the formulations. The decrease in main peak during storage at 25° C. is mainly caused by an increase in acidic variants (around 8%) and a slight increase in basic variants (1-2%). The increase of basic variants after 13 weeks at 25° C. is slightly lower (ca. 1%) with the reference formulations (formulated at pH 6.0) in comparison to the optimized formulations (ca. 2% increase). The lower protein concentration and the primary container have no impact on the charged variants.

TABLE 12
Summary on protein concentration, pH and osmolality results
of the excipient screen II samples after manufacturing

| | | Protein | | |
|---|---|---|---|---|
| | Formulation | concentration | | Osmolality |
| Form. Code | and dosage form | (mg/mL) | pH | (mOsm/kg) |
| GRM0076-02 | 120 mg/mL in PFS | 119 | 5.6 | 318 |
| GRM0077-02 | 30 mg/mL in PFS | 30 | 5.5 | 278 |
| GRM0077-06 | 30 mg/mL in vial | 30 | 5.5 | 278 |
| GRM0077-09 | 30 mg/mL in vial, | 31 | 6.0 | 294 |
| | reference | | | |
| | formulation | | | |
| GRM0076-05 | 120 mg/mL in vial, | 123 | 6.1 | 312 |
| | reference | | | |
| | formulation | | | |

TABLE 13
Summary on visible particle results of initial and stressed excipient
screen II samples

| | | | after | after | | | |
|---|---|---|---|---|---|---|---|
| | | | 1 week | 1 week | after 5 | | |
| | Formulation | | shaking | shaking | freeze/ | after | after |
| | and dosage | | at | at | thaw | 13 weeks | 13 weeks |
| Form. Code | form | Initial | 5° C. | 25° C. | cycles | at 5° C. | at 25° C. |
| GRM0076- | 120 mg/mL | free of | free of | free of | free of | Free of | Free of |
| 02 | in PFS | particles | particles | particles | particles | particles | particles |
| GRM0077- | 30 mg/mL in | free of | free of | free of | free of | Free of | Free of |
| 02 | PFS | particles | particles | particles | particles | particles | particles |
| GRM0077- | 30 mg/mL in | free of | free of | free of | free of | Free of | Free of |
| 06 | vial | particles | particles | particles | particles | particles | particles |
| GRM0077- | 30 mg/mL in | free of | free of | free of | free of | With | With |
| 09 | vial, | particles | particles | particles | particles | many | many |
| | reference | | | | | particles | particles |
| | formulation | | | | | | |
| GRM0076- | 120 mg/mL | free of | free of | free of | free of | Free of | Free of |
| 05 | in vial, | particles | particles | particles | particles | particles | particles |
| | reference | | | | | | |
| | formulation | | | | | | |

### Conclusion

The outcome of the excipient screen II confirmed that the optimized formulations is superior to the reference formulation. The turbidity of the optimized formulations at 120 mg/mL was reduced from greater than 45 FTU to less than 25 FTU, while maintaining the viscosity to less than 15 mPas. A low viscosity is essential in order to enable a commercial-scale production process (up-concentration by ultrafiltration) and to ensure an easy and convenient intravitreal injection (injection forces of less than 20N, in particular less than 15 N). It was demonstrated that the optimized formulation with a viscosity of less than 15 mPas was able to be injected through a 30G injection needle with 5 s injection time with an injection force of less than 5 N.

Furthermore, the optimized formulations at 30 mg/mL remained free of particles, whereas the reference formulations showed visible particles after 13 weeks' storage at 5 and 25° C. In addition, the increase of HMW species was lower in the optimized formulations.

The improved stability behavior of the optimized formulations was observed at protein concentrations of 30 and 120 mg/mL and in a vial, as well as a pre-filled syringe.

In summary, the optimized formulation with protein concentration between 30 and 120 mg/mL containing a histidine-acetate buffer pH 5.5, 25 mM sodium chloride, 7 mM methionine, 160 mM sucrose and 0.04% polysorbate 20 allows a particle-free formulation with low turbidity and viscosity and an improved stability behavior in a vial and a pre-filled syringe.

### Example 7: Safety of Methionine for Use in Ocular Indication (in a Formulation for Intravitreal Application)

Overview of Nonclinical Toxicity Studies with L-Methionine

A toxicity study in cynomolgus monkeys was conducted in which methionine (10 mM) was a component of the vehicle and of the formulated test article CrossMAb VEGFang2-0016 (faricimab). In this study, a total of 12 animals (6 males/6 females) were treated intravitreal with 50 μL/eye two times, 14 days apart. The left eyes were treated with the vehicle (containing 10 mM methionine) and the right eyes were treated with the formulated test article CrossMAb VEGFang2-0016 (faricimab), also containing 10 mM methionine. In this study, no ocular effects of methionine were seen in cynomolgus monkeys.

Three studies (in cynomolgus monkeys and in New Zealand White rabbits) were conducted in which methionine (5-25 mM) was a component of the vehicle, administered intravitreally (also 50 μL/eye) up to six times, 14 days apart. In these studies, no ocular effects of methionine were observed in any of the animals treated with the methionine-containing vehicle.

An overview of the nonclinical toxicity studies is provided in Table 14.

TABLE 14
Summary of Nonclinical Toxicity Studies Containing Methionine (Dosed intravitreally)

| | | | | | |
|---|---|---|---|---|---|
| Species | — | Cynomolgus | NZW rabbit | Cynomolgus | NZW rabbit |
| | | monkey | | monkey | |
| N (m/f) | — | 6M/6F | 3M | 5M/5F | 5M/5F |
| Administration | — | Vehicle in left | Vehicle in | Vehicle in | Vehicle in |
| | | eye, | both eyes | both eyes | both eyes |
| | | formulated | (control | (control | (control |
| | | faricimab in | group) | group) | group) |
| | | right eye | | | |
| N of doses | — | 2 | 1 | 6 | 4 |
| (14 days apart) | | | | | |
| | formulation for | — | — | — | — |
| | CrossMAb | | | | |
| | VEGFang2- | | | | |
| | 0016 | | | | |
| | (faricimab) | | | | |
| Test article | CrossMAb | faricimab | — | — | — |
| | VEGFang2- | | | | |
| | 0016 | | | | |
| | (faricimab) | | | | |
| Vehicle/ | 7 mM | 10 mM | 25 mM | 5 mM | 5 mM |
| control article | methionine | methionine | methionine | methionine | methionine |
| composition | — | — | 5 mM NAT | 1 mM NAT | 1 mM NAT |
| | 20 mM | 20 mM | 20 mM | 20 mM | 20 mM |
| | histidine-acetate | histidine/ | histidine HCl | histidine HCl | histidine HCl |
| | buffer pH 5.5 | histidine-HCl | | | |
| | 160 mM | 50 mM | 240 mM | 240 mM | 240 mM |
| | sucrose | sucrose | sucrose | sucrose | sucrose |
| | 0.04% | 0.04% (w/v) | 0.02% | 0.02% | 0.02% |
| | polysorbate 20 | polysorbate 20 | polysorbate 20 | polysorbate 20 | polysorbate 20 |
| | 25 mM sodium | 100 mM | — | — | — |
| | chloride | sodium | | | |
| | | chloride | | | |

Abbreviations: NAT = N-acetyl tryptophan; NZW = New Zealand white.

### Example 8: Stability

A drug Product batch (120 mg/mL Vegf/Ang2 antibody (Faricimab) in 20 mM L-histidine-acetate pH 5.5, 160 mM sucrose, 25 mM sodium chloride, 7 mM L-methionine, 0.04% polysorbate 20) was filtered through a 0.22 μm sterile filter and filled into clean and sterile 2 mL glass vials with a fill volume of 0.24 mL.

After manufacturing the pH was 5.6, the osmolality 320 mOsm/kg and the protein concentration 120 mg/mL.

Table 15 presents the stability data of Drug Product batch GLI0219-01 during storage at 5° C. Table 16 shows the stability during storage at 25° C.

TABLE 15
Stability data of Vegf-Ang2 antibody (Faricimab) Drug Product
batch during storage at 5° C.

| | | | HMW | Main | Acidic | Basic |
|---|---|---|---|---|---|---|
| Time | Visible | Turbidity | by SEC | peak | peak | peak |
| (wks) | particles | (NTU) | (Area %) | (Area %) | (Area %) | (Area %) |
| 0 | Practi- | 24 | 0.8 | 72.8 | 18.9 | 8.3 |
| | cally | | | | | |
| | free | | | | | |
| | from | | | | | |
| | particles | | | | | |
| 4 | 0 | 22 | 1.2 | 72.2 | 18.9 | 8.9 |
| | particles | | | | | |
| | per | | | | | |
| | 10 vials | | | | | |
| 13 | 0 | 23 | 1.7 | 71.6 | 19.5 | 8.9 |
| | particles | | | | | |
| | per | | | | | |
| | 10 vials | | | | | |
| 26 | 0 | 22 | 2.2 | 71.0 | 19.5 | 9.5 |
| | particles | | | | | |
| | per | | | | | |
| | 10 vials | | | | | |
| 39 | 0 | 24 | 2.4 | 70.6 | 19.8 | 9.6 |
| | particles | | | | | |
| | per | | | | | |
| | 10 vials | | | | | |
| 52 | 0 | 23 | 2.8 | 69.5 | 20.2 | 10.3 |
| | particles | | | | | |
| | per | | | | | |
| | 10 vials | | | | | |
| 65 | 0 | 24 | 3.1 | 68.8 | 20.7 | 10.5 |
| | particles | | | | | |
| | per | | | | | |
| | 10 vials | | | | | |
| 78 | 0 | 24 | 3.3 | 68.3 | 20.8 | 11.0 |
| | particles | | | | | |
| | per | | | | | |
| | 10 vials | | | | | |

TABLE 16
Stability data of Vegf-Ang2 antibody (Faricimab) Drug Product
batch during storage at 25° C.

| | | | HMW | Main | Acidic | Basic |
|---|---|---|---|---|---|---|
| Time | Visible | Turbidity | by SEC | peak | peak | peak |
| (wks) | particles | (NTU) | (Area %) | (Area %) | (Area %) | (Area %) |
| 0 | Practi- | 24 | 1.0 | 72.8 | 18.9 | 8.3 |
| | cally | | | | | |
| | free | | | | | |
| | from | | | | | |
| | particles | | | | | |
| 4 | 0 | 23 | 2.0 | 68.1 | 21.4 | 10.5 |
| | particles | | | | | |
| | per | | | | | |
| | 10 vials | | | | | |
| 13 | 0 | 23 | 2.6 | 59.7 | 28.6 | 11.7 |
| | particles | | | | | |
| | per | | | | | |
| | 10 vials | | | | | |
| 26 | 0 | 23 | 3.0 | 50.0 | 37.2 | 12.8 |
| | particles | | | | | |
| | per | | | | | |
| | 10 vials | | | | | |
| 52 | 0 | 24 | 3.5 | 37.7 | 49.9 | 12.4 |
| | particles | | | | | |
| | per | | | | | |
| | 10 vials | | | | | |

Abbreviations

| Abbreviation | Description |
|---|---|
| His/Ace | Histidine-Acetate buffer |
| His-HCl | Histidine-hydrochloride buffer |
| SE-HPLC | Size exclusion high performance liquid |
| | chromatography |
| IE-HPLC | Ion exchange high performance liquid |
| | chromatography |
| FTU | Formazin turbidity unit |
| HMW | High Molecular Weight Species |
| mPas | milli Pascal second |
| LMW | Low Molecular Weight Species |
| mg/mL | milligram per milliliter |

What is claimed:

1. A liquid pharmaceutical formulation comprising:
120 mg/ml±18 mg/ml of a bispecific antibody against human Angiopoietin-2 and human vascular endothelial growth factor (bispecific anti-VEGF/ANG2 antibody) comprising a constant heavy chain region of human IgG1 subclass,
15 to 35 mM of sodium chloride,
15 to 25 mM of a histidine acetate buffer,
at a pH of 5.5±0.5;
wherein the bispecific anti-VEGF/ANG2 antibody is bivalent and comprises a first antigen-binding site that specifically binds to human vascular endothelial growth factor (VEGF) and a second antigen-binding site that specifically binds to human Angiopoietin-2 (ANG-2), wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14,
iii) the bispecific antibody comprises a constant heavy chain region of human IgG1 subclass comprising the mutations I253A, H310A, and H435A and the mutations L234A, L235A and P329G (numberings according to EU Index of Kabat.

2. The pharmaceutical formulation according to claim 1, wherein the bispecific anti-VEGF/ANG2 antibody is bivalent and comprises a first antigen-binding site that specifically binds to human VEGF and a second antigen-binding site that specifically binds to human ANG-2, wherein
i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.

3. The pharmaceutical formulation according to claim 2, wherein the bispecific anti-VEGF/ANG2 antibody is bivalent and comprises a first antigen-binding site that specifically binds to human VEGF and a second antigen-binding site that specifically binds to human ANG-2,
wherein
iv) in the constant heavy chain region a S354C and T366W mutations are comprised in one CH3 domain and Y349C, T366S, L368A and Y407V mutations are comprised the other CH3 domain (numberings according to EU Index of Kabat).

4. The pharmaceutical formulation according to any one of claims 1 to 3, wherein the bispecific anti-VEGF/ANG2 antibody is bivalent and comprises the amino acid sequences of SEQ ID NO: 17, of SEQ ID NO: 18, of SEQ ID NO: 19, and of SEQ ID NO: 20.

5. The pharmaceutical formulation according to any one of claims 1 to 3, wherein the bispecific anti-VEGF/ANG2 antibody is faricimab.

6. The pharmaceutical formulation according to any one of claims 1 to 5 for intravitreal administration.

7. The pharmaceutical formulation according to any one of claims 1 to 6, wherein the formulation is essentially free of visible particles.

8. The pharmaceutical formulation according to any one of claims 1 to 7, wherein the formulation further comprises
1 to 20 mM of at least one stabilizer.

9. The pharmaceutical formulation according to any one of claims 1 to 7, wherein the formulation further comprises
7.0 mM±2.0 mM methionine.

10. The pharmaceutical formulation according to any one of claims 1 to 9, wherein the formulation further comprises
0.01-0.07% (w/v) of a surfactant.

11. The pharmaceutical formulation according to any one of claims 1 to 9, wherein the formulation further comprises
0.03% to 0.07% (w/v) polysorbate 20.

12. The pharmaceutical formulation according to any one of claims 1 to 11, wherein the formulation further comprises
50-250 mM of a tonicity agent.

13. The pharmaceutical formulation according to any one of claims 1 to 11, wherein the formulation further comprises
160 mM±24 mM sucrose.

14. The pharmaceutical formulation according to any one of claims 1 to 13, wherein the formulation has a viscosity of 20 mPas or less.

15. The pharmaceutical formulation according to any one of claims 1 to 14, wherein the formulation has a turbidity of 30 FTU or less.

16. The pharmaceutical formulation according to any one of claims 1 to 15, wherein the formulation has an ionic strength between 20 and 50.

17. The pharmaceutical formulation according to any one of claims 1 to 16, wherein formulation is a stable formulation.

18. The pharmaceutical formulation according to any one of claims 1 to 17, wherein the high molecular weight species (HMW) content of the bispecific antibody in the pharmaceutical formulation is below 10% after 8 weeks at 25° C. or after 52 weeks at 25° C.

19. The pharmaceutical formulation according to any one of claims 1 to 18, wherein the osmolality of the formulation is 300±100 mOsm/kg.

20. A method of treating an ocular vascular disease comprising administering the pharmaceutical formulation according to any one of claims 1 to 19.

21. The method according to claim 20, wherein the ocular vascular disease is selected from the group consisting of diabetic retinopathy (DR), diabetic macular edema (DME), retinal vein occlusion (RVO), central retinal vein occlusion (CRVO), macular degeneration, wet age-related macular degeneration (wet AMD), retinopathy of prematurity (ROP), neovascular glaucoma, retinitis pigmentosa (RP), retinal angiomatous proliferation, macular telangiectasia, ischemic retinopathy, iris neovascularization, intraocular neovascularization, corneal neovascularization, retinal neovascularization, choroidal neovascularization, and retinal degeneration, in particular from the group consisting of diabetic retinopathy (DR), diabetic macular edema (DME), retinal vein occlusion (RVO), central retinal vein occlusion (CRVO), wet age-related macular degeneration (wet AMD).

22. A method for the preparation of the pharmaceutical formulation of any one of claims 1 to 19
the method comprising the steps of
buffer exchange of the bispecific antibody bulk solution a) against a diafiltration buffer by ultra-filtration and diafiltration or b) by dialysis using a dialysis buffer, the buffers containing a histidine-acetate buffer or a histidine-acetate buffer and sodium chloride, or a histidine-acetate buffer, sodium chloride and methionine, or a histidine-acetate buffer, sodium chloride, methionine and sucrose
concentration of the buffer exchanged bulk solution by ultrafiltration
adjustment of the final composition of the pharmaceutical formulation by addition of stock solutions of the respective excipients or by an appropriate conditioning buffer and homogenization of the liquid pharmaceutical formulation is homogenized by mixing.

23. A vial comprising the pharmaceutical formulation of any one of claims 1 to 19.

24. A prefilled syringe comprising the pharmaceutical formulation of any one of claims 1 to 19.

25. A lyophilized form of the liquid pharmaceutical formulation of any one of claims 1 to 19.


SEQUENCE LISTING (25 sequences)

SEQ ID NO: 1 - protein, 14 aa, synthetic construct
  REGION 1..14: note = heavy chain CDR3H, <VEGF>
YPYYYGTSHWYFDV

SEQ ID NO: 2 - protein, 17 aa, synthetic construct
  REGION 1..17: note = heavy chain CDR2H, <VEGF>
WINTYTGEPTYAADFKR

SEQ ID NO: 3 - protein, 5 aa, synthetic construct
  REGION 1..5: note = heavy chain CDR1H, <VEGF>
HYGMN

SEQ ID NO: 4 - protein, 9 aa, synthetic construct
  REGION 1..9: note = light chain CDR3L, <VEGF>
QQYSTVPWT

SEQ ID NO: 5 - protein, 7 aa, synthetic construct
  REGION 1..7: note = light chain CDR2L, <VEGF>
FTSSLHS

SEQ ID NO: 6 - protein, 11 aa, synthetic construct
  REGION 1..11: note = light chain CDR1L, <VEGF>
SASQDISNYLN

SEQ ID NO: 7 - protein, 123 aa, synthetic construct [sequence omitted]

SEQ ID NO: 8 - protein, 107 aa, synthetic construct [sequence omitted]

SEQ ID NO: 9 - protein, 20 aa, synthetic construct
  REGION 1..20: note = heavy chain CDR3H, <ANG-2>
SPNPYYYDSSGYYYPGAFDI

SEQ ID NO: 10 - protein, 17 aa, synthetic construct
  REGION 1..17: note = heavy chain CDR2H, <ANG-2>
WINPNSGGTNYAQKFQG

SEQ ID NO: 11 - protein, 5 aa, synthetic construct
  REGION 1..5: note = heavy chain CDR1H, <ANG-2>
GYYMH

SEQ ID NO: 12 - protein, 11 aa, synthetic construct
  REGION 1..11: note = light chain CDR3L, <ANG-2>
QVWDSSSDHWV

SEQ ID NO: 13 - protein, 7 aa, synthetic construct
  REGION 1..7: note = light chain CDR2L, <ANG-2>
DDSDRPS

SEQ ID NO: 14 - protein, 11 aa, synthetic construct
  REGION 1..11: note = light chain CDR1L, <ANG-2>
GGNNIGSKSVH

SEQ ID NO: 15 - protein, 129 aa, synthetic construct
  REGION 1..129: note = heavy chain variable domain VH, <ANG-2>
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSS

SEQ ID NO: 16 - protein, 110 aa, synthetic construct
  REGION 1..110: note = light chain variable domain VL, <ANG-2>
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSS

SEQ ID NO: 17 - protein, 453 aa, synthetic construct
  REGION 1..453: note = Heavy chain 1 of <VEGF-ANG-2> CrossMAb IgG1 with AAA mutations and P329G LALA mutations (VEGFang2-0016)
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNAYTQKSLSLSPGK

SEQ ID NO: 18 - protein, 463 aa, synthetic construct
  REGION 1..463: note = Heavy chain 2 of <VEGF-ANG-2> CrossMAb IgG1 with AAA mutations and P329G LALA mutations (VEGFang2-0016)
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPGK

SEQ ID NO: 19 - protein, 214 aa, synthetic construct
  REGION 1..214: note = Light chain 1 of <VEGF-ANG-2> CrossMAb IgG1 with AAA mutations and P329G LALA mutations (VEGFang2-0016)
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 213 aa, synthetic construct
  REGION 1..213: note = Light chain 2 of <VEGF-ANG-2> CrossMAb IgG1 with AAA mutations and P329G LALA mutations (VEGFang2-0016)
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 21 - protein, 107 aa, Homo sapiens [sequence omitted]

SEQ ID NO: 22 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 330 aa, Homo sapiens [sequence omitted]

SEQ ID NO: 24 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 25 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF